(19) United States
(12) Patent Application Publication
(10) Pub. No.: US 2016/0332971 A1
(43) Pub. Date: Nov. 17, 2016

(54) ARYLQUINOLINE, ARYLQUINOLONE AND ARYLTHIOQUINOLONE DERIVATIVES AND USE THEREOF TO TREAT CANCER

(71) Applicant: UNIVERSITY OF KENTUCKY RESEARCH FOUNDATION, Lexington, KY (US)

(72) Inventors: David S. WATT, Lexington, KY (US); Chunming LIU, Lexington, KY (US); Vivek M. RANGNEKAR, Lexington, KY (US); Vitaliy M. SVIRIPA, Lexington, KY (US); Ravshan BURIKHANOV, Lexington, KY (US); Wen ZHANG, Lexington, KY (US)

(21) Appl. No.: 15/152,185

(22) Filed: May 11, 2016

Related U.S. Application Data
(60) Provisional application No. 62/162,410, filed on May 15, 2015.

(51) Int. Cl. C07D 215/38; C07D 215/48; G01N 33/50; C07D 471/04

(52) U.S. Cl. CPC C07D 215/38; C07D 471/04; C07D 215/48; G01N 33/5011; G01N 2333/705; G01N 2333/47

(57) ABSTRACT

Arylquinoline derivatives and their use for treating cancer or cancer metastasis is disclosed. The compounds of the subject technology promote cells to secrete a pro-apoptotic tumor suppressor, i.e., prostate apoptosis response-4 (Par-4), which in turn promote apoptosis in cancer cells or metastatic cells.

### CROSS-REFERENCE TO RELATED APPLICATION

This application claims the benefit of U.S. Provisional Application No. 62/162,410 filed May 15, 2015 the entire disclosure of which is hereby incorporated by reference herein.

### STATEMENT REGARDING FEDERALLY SPONSORED RESEARCH

This work was supported by NIH grant R21 CA139359 and CA172379; and NIH/NCI R01 CA60872 (to VMR). The government has certain rights in the subject technology.

### TECHNICAL FIELD

The present invention relates to compounds that treat cancer and/or treat or prevent cancer metastasis. In particular, the subject technology is directed to arylquinoline, arylquinolone and arylthioquinolone derivatives that promote cells to secrete a pro-apoptotic tumor suppressor, such as prostate apoptosis response-4 (Par-4), which promotes apoptosis in cancer cells or metastatic cells.

### BACKGROUND

Lung cancer is the most frequently diagnosed cancer and the leading cause of cancer-related deaths in the world. The most common alterations in lung cancer include activating mutations in ras genes and inactivating mutations in the p53 gene. Lung tumor cells with p53 mutations or deletions often develop resistance to chemotherapy and radiation therapy, leading ultimately to the death of the patients. Notably, such p53-deficient cancer cells are susceptible to apoptosis by the proapoptotic tumor suppressor, Par-4.

Par-4 is a tumor suppressor protein that induces apoptosis in diverse cancer cells but not in normal cells. Par-4 is ubiquitously expressed in normal cells and tissues, but is sequestered by an intermediate filament protein, vimentin, and hence, circulating levels of Par-4 are generally low. If it were secreted by normal cells at appreciably higher levels than normal, certain cancer cells would be susceptible to its effects. Extracellular Par-4 binds a receptor GRP78, which appears only on the cancer cell surface, and induces apoptosis by caspase-dependent mechanisms. In contrast, normal cells express low to undetectable levels of basal or inducible cell-surface GRP78 and are resistant to apoptosis by extracellular Par-4.

Arylquinoline and analog compounds have been disclosed for use in the treatment of cancer. See WO2015/077550 and Burikhanov et al. “Arylquins target vimentin to trigger Par-4 secretion for tumor cell apoptosis”, Nature Chemical Biology, 2014:10(11):924-926.

However, there is a continuing need for compounds that are Par-4 secretagogues and promote the secretion of Par-4 which in turn promotes apoptosis in cancer cells and metastatic cells.

### SUMMARY OF THE DISCLOSURE

Advantages of the subject technology include arylquinoline derivatives and compositions for the treatment of cancer or for the treatment or inhibition of cancer metastasis in a subject in need thereof comprising administering to the subject an effective amount of the compound or a pharmaceutically acceptable salt thereof or a composition thereof.

Other advantages of the subject technology include compounds for use in promoting the secretion of Prostate Apoptosis Response-4 (PAR-4) from cells or for use in promoting apoptosis of a cancer cell in a subject comprising administering to the subject an effective amount of an arylquinoline derivative or a pharmaceutically acceptable salt thereof or a composition thereof.

In one aspect of the subject technology, the arylquinoline derivative is a compound according to Formula (I):

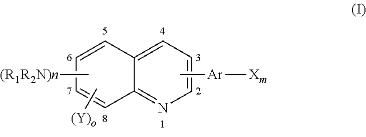

or a pharmaceutically acceptable salt thereof; wherein n is 1, 2, or 3, for each NR₁R₂, R₁ and R₂ are independently H, alkyl, alkoxy, aryl, heteroaryl; Ar is aryl or heteroaryl, which can be further substituted with halogen, amino, alkylamino, dialkylamino, arylalkylamino, N-oxides of dialkylamino, trialkylammonium, mercapto, alkylthio, alkanoyl, nitro, nitrosyl, cyano, alkoxy, alkenyloxy, aryl, heteroaryl, sulfonyl, sulfonamide, CONR₃R₄, NR₃CO(R₄), NR₃COO(R₄), NR₃CONR₄R₅ where R₃, R₄, R₅, are independently, H, alkyl, aryl, heteroaryl or a fluorine; X represents halogen; m is 1, 2, 3, 4, or 5; o is 1, 2, 3 or 4; and each Y, e.g., Y₁, Y₂, and/or Y₃ is independently a halogen, alkoxy (e.g., R₆—O—), alkylthio (e.g., R₆—S—) where R₆ is alkyl, a substituted or unsubstituted heterocycle, e.g., a nitrogen-containing heterocycle such as morpholino, piperazinyl, N-methylpiperazinyl, etc.

In one aspect of the present disclosure, n is 1 or 2; for each NR₁R₂, R₁ and R₂ are independently H or a lower alkyl; m is 1 to 3 and X is selected from fluorine or chlorine, e.g. X is one, two or three fluorine substituents, or X is one, two or three chlorine substituents, or X represents at least one fluorine and at least one chlorine on Ar; Ar is phenyl or pyridinyl, diazinyl, pyrimidinyl, oxazolyl or o is 1; and Y is chloro, fluoro, a C₁-C₆ alkoxy, a C₁-C₆ alkylthio or a substituted or unsubstituted nitrogen-containing heterocycle such as morpholino, piperazinyl, N-methylpiperazinyl, etc. In various embodiments, the compound of Formula (I) includes wherein the substituent at the C-2 position of the quinoline ring is either NR₁R₂, or Y, the substituent at C-3 is phenyl or pyridinyl, diazinyl, pyrimidinyl, oxazolyl or imidazolyl; the substituent at C-4 is H; the substituent at C-5 is H or Y, e.g., a halogen; the substituent at C-6 is H or Y, e.g., a halogen; the substituent at C-7 is H, NR₁R₂, or Y; the substituent at C-8 is H; provided that there is at least one NR₁R₂ at C-2 or C-7 and at least one Y at C-2, C-5, C-6 or C-7.

In another aspect, the subject technology relates to compounds where the Ar-Xm group is located at the 3 position of the quinoline, quinolone or thioquinolone ring and Ar is a phenyl group such as shown in formulas (II) or (III):

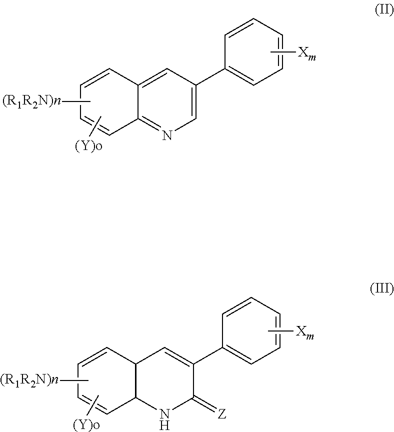

or a pharmaceutically acceptable salt thereof in Formulas (II) and (III), Z is O or S; and n, R₁, R₂, m, X, o and Y are as defined for the compound of Formula (I) including the various embodiments thereof. The compounds of Formula (III) are arylquinolone derivatives and arylthioquinolone derivatives when Z is O or S, respectively, and are useful in the same manner as the compounds according to Formula (I). For ease of reference, the compounds of Formulas (I), (II), (III) will be referred to herein as arylquinoline derivatives.

Another aspect of the subject technology includes a biotinylated derivative or other detectably labeled alternative of each of Formulas (I), (II) and (III) and their various embodiments.

In another aspect, the subject technology relates to pharmaceutical compositions of arylquinoline derivatives, e.g., one or more compounds of Formula (I), Formula (II) and/or Formula (III), and/or one or more pharmaceutically acceptable salts thereof; in combination with a pharmaceutically acceptable additive, e.g., a pharmaceutically acceptable carrier or excipient. In one aspect of the present disclosure, the pharmaceutical compositions comprise an effective amount of at least one compound of Formula (I), (II) or (III) or its pharmaceutically acceptable salt.

In another aspect, the subject technology relates to a method of treating cancer and/or treating or inhibiting cancer metastasis in a subject, e.g., a human. In an embodiment relating to this aspect, a therapeutically effective amount of one or more arylquinoline derivatives, pharmaceutical salts and/or compositions thereof is administered to a subject in need thereof to treat cancer and/or treat or inhibit cancer metastasis in the subject.

In another aspect, the subject technology relates to a method for promoting secretion of Prostate Apoptosis Response-4 (Par-4) from cells or promoting apoptosis of cancer cells in a subject in need thereof by administering to the subject an effective amount of one or more arylquinoline, arylquinolone or arylthioquinolone derivatives or compositions in accordance with the subject technology.

In another aspect, the subject technology relates to a method for screening for compounds that inhibit vimentin binding to PAR-4, comprising exposing a solution including vimentin and PAR-4 to a test compound and detecting the level of vimentin-PAR-4 complex formation by Western blot analysis, for example.

In another aspect, the subject technology relates to a kit which includes the compounds of the subject technology. In an embodiment related to this aspect, the kit includes one or more compounds of Formula (I), (II) and/or (II). In another embodiment, the kit includes one or more other therapeutic compounds for use in combination therapies.

Additional advantages of the subject technology will become readily apparent to those skilled in this art from the following detailed description, wherein only the preferred embodiment of the disclosure is shown and described, simply by way of illustration of the best mode contemplated of carrying out the disclosure. As will be realized, the disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is an exemplary illustration for synthesis of certain arylquinolines with variations in the 3-aryl substituent. Reagents: a, arylacetonitrile XC₆H₄CH₂CN, tert-BuOK, DMF, 90° C., 3-4 h; 2-(2′-fluorophenypacetyl chloride, Et₃N, reflux, 2 h and K₂CO₃, DMF, 90° C., 4 h.

FIG. 2 is another illustration for synthesis of certain arylquinolines with variations in the quinoline, quinolone or thioquinolone rings. Reagents: a, 2-(2′-fluorophenyl)acetyl chloride, Et₃N, reflux, 2h and K₂CO₃, DMF, 90° C., 4h; h, 2,4-bis(4-methoxyphenyl)-2,4-dithioxo-1,3,2,4-dithiadiphosphetane (Lawesson's reagent), dioxane, reflux, 5 h; c, CH₃I, K₂CO₃, DMF; d, POCl₃, reflux. 1 h; e, N-rnethylpiperazine or morpholine, reflux, 12 h; j; 2-FC₆H₄CH₂CN, tert-BuOK, DMF, 90° C., 3-4 h.

FIG. 3 shows quantitation of Par-4 secretion by 500 nM arylquinoline 1a. Calibration curve developed using fixed amounts (0.05-5 ng) of recombinant TRX-Par-4. Calculation for Par-4 concentration using arylquinoline 1a: band density of 40 μL of Par-5 containing solution (lane marked 1a) was 95, which corresponded to 0.42 ng of Par-4. Therefore the concentration was 0.82 ng/40 μL or 540 pM, In the same fashion, calculation for Par-4 concentration using vehicle (v) was 280 pM.

### DETAILED DESCRIPTION OF THE DISCLOSURE

In general, the subject technology relates to Par-4 secretagogues that induce the release of Par-4 from normal cells thereby triggering the paracrine apoptosis of cancer cells. In accordance with the subject technology, certain arylquinoline, arylquinolone and arylthioquinolone derivatives have been identified as Par-4 secretagogues which induce or promote Par-4 secretion at low (nanomolar) concentrations from both normal lung fibroblasts and epithelial cells. The arylquinoline, arylquinolone and arylthioquinolone derivatives and their pharmaceutically acceptable salts and compositions are useful for the treatment of colorectal cancer, prostate cancer, brain cancer, liver cancer, breast cancer and lung cancer. In particular, the arylquinoline, arylquinolone and arylthioquinolone derivatives and their pharmaceutically acceptable salts and compositions are particularly useful in the treatment of lung cancer and prostate cancer.

The Par-4 gene was first identified in 1994 in prostate cancer cells undergoing apoptosis. This gene encodes a pro-apoptotic protein, Prostate Apoptosis Response-4 or Par-4, which is remarkably effective in inducing cancer cell apoptosis and tumor regression in animal models. Par-4 does not affect normal cells, Par-4 protein is secreted in cell culture-conditioned medium (CM) or systemically in mice by normal cells, and extracellular Par-4 binds to its receptor GRP78 on the cancer cell surface and induces apoptosis. Normal cells express low to undetectable levels of cell surface GRP78 and are resistant to apoptosis by extracellular Par-4.

Par-4 induces apoptosis in many types of cancer cells. For cancer cells that may be resistant to direct apoptosis by Par-4, overexpression of Par-4 in these cells renders them supersensitive to a broad range of apoptotic insults, including chemotherapeutic agents, TNF, or ionizing radiation. Applicants have also found that GRP78 levels can be increased on the surface of diverse cancer cells to overcome Par-4-resistance by inhibition of NF-κB activity, which is usually elevated in most cancer cells. Therefore, the arylquinoline, arylquinolone and arylthioquinolone derivatives of the subject technology can be administered either alone or in combination with a second active ingredient such as a chemotherapeutic agent or an NF-κB inhibitor for treating cancer or cancer metastasis.

As the baseline levels of Par-4 secreted by normal cells are generally inadequate to cause massive apoptosis in cancer cell cultures, secretogogues that bolster the release of Par-4 would constitute an important therapeutic advance. Certain arylquinoline Par-4 secretagogues are disclosed in PCT/US2014/066796.

The present disclosure relates to arylquinoline, arylquinolone and arylthioquinolone derivatives that can also promote the desired secretion of Par-4 in vitro and in vivo by selectively targeting an intermediate filament protein, vimentin. The arylquinoline, arylquinolone and arylthioquinolone derivatives of the present disclosure include compounds according to Formula (I):

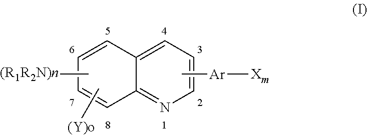

wherein n is 1, 2, or 3, for each NR₁R₂, R₁ and R₂ are independently H, alkyl, alkoxy, aryl, heteroaryl; Ar is aryl, e.g., phenyl, naphthyl, and heteroaryl, e.g., pyridyl, pyrolidyl, piperidyl, pyrimidyl, indolyl, thienyl, which can he further substituted with halogen, amino, alkylamino, dialkylamino, arylalkylamino, N-oxides of dialkylamino, trialkylammonium, mercapto, alkylthio, alkanoyl, nitro, nitrosyl, cyano, alkoxy, alkenyloxy, aryl, heteroaryl, sulfonyl, sulfonamide, CONR₃R₄, NR₃CO(R₄), NR₃COO(R₄), NR₃CONR₄R₅ where R₃, R₄, R₅, are independently, H, alkyl, aryl, heteroaryl or a fluorine; X represents halogen, e.g., a fluorine, chlorine, bromine, or iodine substituent; m is 1, 2, 3, 4, 5; o is 1, 2, 3 or 4; and each Y, e.g., Y₁, Y₂, and/or Y₃ is independently a halogen, alkoxy (e.g., R₆—O—), alkylthio (e.g., R₆—S—) where R₆ is alkyl, a substituted or unsubstituted heterocycle, e.g., a nitrogen-containing heterocycle such as morpholino, piperazinyl, N-methylpiperazinyl, etc. This embodiment also includes pharmaceutically acceptable salts of Formula (I).

In one aspect of the present disclosure, n is 1 or 2; for each NR₁R₂, R₁ and R₂ are independently H or a lower alkyl; m is 1 to 3 and X is selected from fluorine or chlorine, e.g. X is one, two or three fluorine substituents, or X is one, two or three chlorine substituents, or X represents at least one fluorine and at least one chlorine on Ar; Ar is phenyl or pyridinyl, diazinyl, pyrimidinyl, oxazolyl or imidazolyl; o is 1; and Y is chloro, fluoro, a C₁-C₆ alkoxy, a C₁-C₆ alkylthio, or a substituted or unsubstituted nitrogen-containing heterocycle.

In various embodiments, the compound of Formula (I) includes wherein the substituent at the C-2 position of the quinoline ring is either NR₁R₂, or Y, the substituent at C-3 is phenyl or pyridinyl, diazinyl, pyrimidinyl, oxazolyl or imidazolyl; the substituent at C-4 is H; the substituent at C-5 is H or Y, e.g., a halogen; the substituent at C-6 is H or Y, e.g., a halogen; the substituent at C-7 is H, NR₁R₂, or Y; the substituent at C-8 is H; provided that there is at least one NR₁R₂ at C-2 or C-7 and at least one Y at C-2, C-5, C-6 or C-7. In this embodiment, for each NR₁R₂, R₁ and R₂ are independently H or a lower alkyl; m is 1 to 3 and X is selected from fluorine or chlorine; and Y is chloro, fluoro, a C₁-C₆ alkoxy, a C₁-C₆ alkylthio or a substituted or unsubstituted nitrogen-containing heterocycle such as morpholino, piperazinyl, N-methylpiperazinyl, etc.

In another aspect, the Ar-Xm group of the arylquinoline derivative is located at the 3 position of the quinoline ring and Ar is a phenyl group such as shown in formulas (II) or (III):

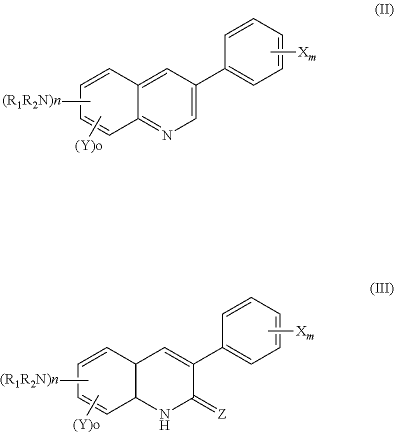

or a pharmaceutically acceptable salt thereof; wherein Z is O or S; and n, R₁, R₂, m, X, o and Y are as defined for the compound of Formula (I) including the various embodiments thereof.

In one aspect of this embodiment, the compounds of Formula (II) and Formula (III) have the substituent at the C-2 position of the quinoline ring is either NR₁R₂, or Y, the substituent at C-3 is phenyl or pyridinyl, diazinyl, pyrimidinyl, oxazolyl or imidazolyl; the substituent at C-4 is H; the substituent at C-5 is H or Y; the substituent at C-6 is H or Y; the substituent at C-7 is H, NR₁R₂, or Y; the substituent at C-8 is H; provided that there is at least one NR₁R₂ at C-2 or C-7 and at least one Y at C-2, C-5, C-6 or C-7. In this embodiment, for each NR₁R₂, R₁ and R₂ are independently H or a lower alkyl; m is 1, 2 or 3; X is selected from fluorine or chlorine; and Y is chloro, fluoro, a C₁-C₆ alkoxy, a C₁-C₆ alkylthio or a substituted or unsubstituted nitrogen-containing heterocycle such as morpholino, piperazinyl, N-methylpiperazinyl, etc. Preferably the phenyl ring has at least one fluoro or chloro group in the ortho- (or C-2′) position.

Another aspect of the subject technology includes a biotinylated derivative or other detectably labeled alternative of each of Formulas (I), (II) and (III) and their various embodiments,

Nanomolar levels of 3-arylquinolines, which we called “arylquins”, promote the enhanced release of Par-4 from vimentin in normal cells in vitro and in vivo. Burikhanov, et al., Arylquins targets vimentin to trigger Par-4 secretion for tumor cell apoptosis Nature Chem Biol 2014, 10(/1):924-6. Once in circulation, this extracellular Par-4 selectively binds to a receptor, Glucose-regulated protein-78 (GRP78), that appears only on the surface of cancer cells, and this binding event induces intracellular caspase-driven apoptosis. Because various cancers, including advanced prostate cancer, express GRP78, any agent that promotes the enhanced secretion of Par-4 from normal cells, which vastly outnumber cancer cells, represents an attractive therapeutic approach, Because vimentin is also a key component of the epithelial-mesenchymal transition (EMT) necessary for metastasis in diverse cancers, the identification of vimentin as the target for arylquinolines is also consistent with arylquinolines and their derivatives functioning as antineoplastic agents.

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

To facilitate an understanding of the present subject technology, a number of terms and phrases are defined below:

The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

The term “treat” and “treatment” refer to both therapeutic treatment and prophylactic, inhibition or preventative measures, wherein the object is to inhibit, prevent or slow down (lessen) an undesired pathological change or disorder, such as the development or spread of cancer. For purpose of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, “treatment” can include a qualitative or quantitative reduction (e.g., by at least 10%, 20%, 30%, 40%, 50%, 60%, 70%, 80%, 90%, 95%, or more) in the tumor or metastases size or reduce, inhibit, or prevent metastatic growth. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the subject technology that (i) treats, inhibits, or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may be reduce the number of cancer cells; reduce the tumor size; inhibit (i.e., slow to some extent and preferably stop) cancer cell infiltration into peripheral organs; inhibit (i.e., slow to some extent and preferably prevent or stop) tumor metastasis; inhibit, to some extent, tumor growth; and/or relieve to some extent one or more of the symptoms associated with the cancer. To the extent the drug may prevent growth, inhibit, and/or kill existing cancer cells, it may be cytostatic and/or cytotoxic, For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatome, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “prodrug” as used in this application refers to a precursor or derivative form of a compound of the disclosure that may be less cytotoxic to cells compared to the parent compound or drug and is capable of being enzymatically or hydrolytically activated or converted into the more active parent form. The prodrugs of this disclosure include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, enzymatic cleavage, and the like, of the administered compound.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀) for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” as used herein also includes halo-substituted alkyls.

Unless the number of carbons is otherwise specified, “lower alkyl” refers to an alkyl group, as defined above, but having from one to about ten carbons (C₁-C₁₀), e.g., from one to about six carbon atoms (C₁-C₆) in its backbone structure. Likewise, “lower alkenyl” “loweralkyl, “lower amino”, “lower alkynyl”, etc. have similar chain lengths.

Therapeutic Agents

Disclosed herein are arylquinoline derivatives, i.e., compounds of Formula (I), (II) and (III), and their use in treating cancer cells or in treating, or inhibiting metastatic cells. Such compounds of the subject technology are Par-4 secretagogues, i.e., promote secretin of Par-4 from cells, which promote apoptosis in cancer cells or metastatic cells. Thus, in an embodiment, the compounds of the subject technology are useful in treating cancers including, but not limited to, colorectal cancer, liver cancer, breast cancer and lung cancer.

### Synthesis

The compounds of the subject technology, including compounds of Formula (I) to Formula (II), may be prepared by methods disclosed herein or any other method known in the art. One of ordinary skill in the art will know how to modify procedures to obtain the analogs of the subject technology. In addition, compounds may be prepared using the methods described below and the Examples or modified versions thereof.

The subject technology also encompasses biotinylated derivatives of the arylquinoline derivatives. Such biotinylated derivatives are useful in identifying the molecular target for these agents. Compounds encompassed by Formulas (I), (II) and (III) can be synthesized and converted to biotinylated derivatives.

In certain embodiments of the subject technology, the arylquinoline, arylquinolone and arylthioquinolone derivatives of the disclosure, or a pharmaceutically acceptable salt, solvate, hydrate or prodrug thereof, inhibit the growth or spread of cancer cells by promoting apoptosis in them.

Metabolites of Compounds of the Disclosure

Also falling within the scope of this disclosure are the in vivo metabolic products of Formulas (I) to (II) described herein. Such products may result for example from the oxidation, reduction, hydrolysis, amidation, deamidation, esterification, deesterification, enzymatic cleavage, and the like, of the administered compound.

Accordingly, the disclosure includes metabolites of compounds of Formulas (I) to (II), including compounds produced by a process comprising contacting a compound of this disclosure with a mammal for a period of time sufficient to yield a metabolic product thereof.

Metabolite products typically are identified by preparing a detectably labeled, for example a radiolabeled (e.g., C or H isotope) compound of the disclosure, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are detectably labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies, which are well known to those skilled in the art. The metabolite products, so long as they are not otherwise found in vivo, are useful in diagnostic assays for therapeutic dosing of the compounds of the disclosure.

Prodrugs of the Compounds of the Disclosure

In addition to compounds of the subject technology, the disclosure also includes pharmaceutically acceptable prodrugs of such compounds. Prodrugs include compounds wherein an amino acid residue, or a polypeptide chain of two or more (e.g., two, three or four) amino acid residues, is covalently joined through an amide or ester bond to a free amino, hydroxy or carboxylic acid group of a compound of the subject technology. The amino acid residues include but are not limited to the 20 naturally occurring amino acids commonly designated by three letter symbols and also includes phosphoserine, phosphothreonine, phosphotyrosine, 4-hydroxyproline, hydroxyzine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, garnma-aminobutyric acid, citrulline, homocysteine, homoserine, methyl-alanine, para-benzoylphenylalanine, phenylglycine, propargylglycine, sarcosine, methionine sulfone and tert-butylglycine.

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundaaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., journal of Pharmaceutical Sciences, 77:285 (1988); and e) N. Kakeya, et al., Chem. Pharm. Bull., 32:692 (1984), each of which is specifically incorporated herein by reference.

Pharmaceutical Compositions

The subject technology also encompasses pharmaceutical compositions comprising at least one arylquinoline derivatives, e.g., one or more compounds of Formula (I), (II), and/or Formula (III) and/or one or more pharmaceutically acceptable salts thereof, in combination with a pharmaceutical additive, e.g., a phamaceutical carrier or excipient. In one embodiment of the subject technology, the pharmaceutical compositions comprise an effective amount of at least one such compound. In another embodiment, the pharmaceutical composition comprises one or more compounds of Formula (III) or salt thereof, and a pharmaceutically acceptable additive.

While it may be possible for compounds of the subject technology to be administered as the raw chemical, it is preferable to present them as a pharmaceutical composition. According to a further aspect, the subject technology provides a pharmaceutical composition comprising a compound or mixture of compounds of Formula (I) to Formula (II) or a pharmaceutically acceptable salt, solvate, hydrate, prodrug or metabolite thereof, together with one or more pharmaceutical carrier, excipient or additive and optionally one or more other therapeutic ingredients.

To prepare the pharmaceutical compositions, a therapeutically effective amount of one or more of the arylquinoline derivatives according to the subject technology may be intimately admixed with a pharmaceutically acceptable carrier according to conventional pharmaceutical compounding techniques to produce a dose. A carrier may take a wide variety of forms depending on the form of preparation desired for administration, e.g., oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

In one embodiment, the compositions are prepared with carriers that will protect the active compound(s) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The pharmaceutically acceptable carrier may take a wide variety of forms, depending on the route desired for administration, for example, oral or parenteral (including. intravenous). Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by standard aqueous or nonaqueous techniques. Oral and parenteral sustained release dosage forms may also be used.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension. Other methods of preparation well known by those of ordinary skill may also be used in this aspect of the subject technology.

In an embodiment, the composition of the subject technology enables sustained, continuous delivery of a compound of Formula (I) to Formula (II) or a pharmaceutically acceptable salt, solvate, hydrate, prodrug or metabolite thereof, to tissues adjacent to or distant from an administration site. The biologically-active agent is capable of providing a local or systemic biological, physiological or therapeutic effect. For example, a compound of Formula (I) to Formula (II) or a pharmaceutically acceptable salt, solvate, hydrate, prodrug or metabolite thereof, may act to kill cancer cells, or cancer stem cells or to control or suppress tumor growth or metastasis, among other functions.

Formulations and Dosages for Administration

Pharmaceutical formulations based upon arylquinoline, arylquinolone and arylthioquinolone derivatives of the subject technology comprise at least one of the compounds of Formula (I) to Formula (III) or a pharmaceutically acceptable salt, solvate, hydrate, prodrug or metabolite thereof, in a therapeutically effective amount for treating neoplasia, cancer and other diseases and conditions that may benefit from induced Par-4 secretion, optionally in combination with a pharmaceutically acceptable additive, carrier and/or excipient. One of ordinary skill in the art will recognize that a therapeutically effective amount of one of more compounds according to the subject technology will vary with the condition to be treated, its severity, the treatment regimen to be employed, the pharmacokinetics of the agent used, as well as the patient (animal or human) treated.

Exemplary formulations are well known to those skilled in the art, and general methods for preparing them are found in any standard pharmacy school textbook, for example, Remington: THE SCIENCE AND PRACTICE OF PHARMACY, 21st Ed., Lippincott. The formulations of the subject technology may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations are well known to those skilled in the art, and general methods for preparing them are found in any standard pharmacy school textbook, for example, Remington: THE SCIENCE AND PRACTICE OF PHARMACY, 21st Ed., the entire disclosure of which is incorporated herein by reference.

The concentration of active compound of the subject technology, i.e., at least one of the compounds of Formula (I) to Formula (III) or a pharmaceutically acceptable salt, solvate, hydrate, prodrug or metabolite thereof, in the drug composition will depend on absorption, distribution, inactivation, and excretion rates of the drug as well as other factors known to those of skill in the art. It is to be noted that dosage values will also vary with the severity of the condition to be alleviated. The composition may be administered at once, or may be divided into a number of smaller doses to be administered at varying intervals of time.

Oral compositions will generally include an inert diluent or an edible carrier, They may be enclosed in gelatin-capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following non-limiting ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; glidant such as colloidal silicon dioxide; a sweetening agent such as sucrose or saccharin; or a flavoring agent such as peppermint, methyl salicylate, or fruit flavoring. When the dosage unit form is a capsule, it can contain, in addition to any of the above, a liquid cam r such as fatty oil. In addition, dosage unit forms can contain various other materials which modify the physical form of the dosage unit, for example, coatings of sugar, shellac, or enteric agents.

The tablets, for example, may optionally be coated or scored and may be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington: The Science and Practice of Pharmacy, 21st Ed.

The active compound may also be administered as a component of an elixir, suspension, syrup, wafer or the like. Syrup may contain, in addition to the active compounds, sucrose or fructose as a sweetening agent and certain preservatives, dyes and colorings and flavors.

In certain embodiments of the subject technology, the arylquinoline, arylquinolone and arylthioquinolone derivatives are formulated as admixture with a pharmaceutically acceptable carrier, excipient or additive. In certain pharmaceutical dosage forms, the pro-drug form of the compounds may be preferred. One of ordinary skill in the art will recognize how to readily modify the present compounds to pro-drug forms to facilitate delivery of active compounds to a targeted site within the host organism or patient. The practitioner also will take advantage of favorable pharmacokinetic parameters of the pro-drug forms, where applicable, in delivering the present compounds to a targeted site within the host organism or patient to maximize the intended effect of the compound.

Pharmaceutical compositions containing any of the compounds of Formula (I) to Formula (III) or a pharmaceutically acceptable salt, solvate, hydrate, prodrug or metabolite thereof, may be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 0.0001 mg per day to about 2 mg per day, or about 0.1 mg per day to about 100 mg per day, or about 10 mg per day to about 1000 mg per day, or from about 100 mg per day to about 10000 mg per day, or from about 5 mg per day to about 100 mg per day, to about 50 mg per day or to about 250 mg per day. In some embodiments, the total daily dose may range from about 1 mg per day to about 50 mg per day, or about 10 mg per day to about 500 mg per day. It is further recommended that children, patients over 65 years old, and those with impaired renal or hepatic function, initially receive low doses, and that the dosage be titrated based on individual responses and/or blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

Alternatively, the maximum safe starting dose of the compounds of the subject technology for use in initial clinical trials in adults may be determined by following, for example, the FDA guidelines for estimating maximum safe dosage. These guidelines provide guidance for using the dosages used in animal studies to extrapolate safe dosage for use in human trials. See Guidance for Industry, Estimating the Maximum Safe Starting Dose in Initial Clinical Trials for Therapeutics in Adult Healthy Volunteers, Food and Drug Administration, Center for Drug Evaluation and Research (CDER), July 2005.

In an embodiment, the amount of compound included within therapeutically effective formulations of the subject technology is an effective amount for treating cancer or cancer metastasis by promoting apoptosis in the cancer cells. In general, a therapeutically effective amount of the present preferred compound in dosage form usually ranges from slightly less than about 0.0001 mg/kg. to about 0.003 mg/kg or about 0.0025 mg/kg. to about 2.5 g/kg, and in certain embodiments about 0.025 mg/kg to about 5 mg/kg or about 0.25 mg/kg to about 100 mg/kg or about 2.5 mg/kg to about 250 mg/kg or about 25 mg/kg to about 500 mg/kg or considerably more, depending upon the compound used, the condition being treated and the route of administration, although exceptions to this dosage range may be contemplated by the subject technology. In some embodiments, arylquinoline, arylquinolone and arylthioquinolone derivatives of the subject technology are administered in amounts ranging from about 0.0001 mg/kg to about 1000 mg/kg.

The active compound of the subject technology, i.e., at least one of the compounds of Formula (I), (II), or (II) or a pharmaceutically acceptable salt, solvate, hydrate, prodrug or metabolite thereof, is included in the pharmaceutically acceptable carrier or diluent in an amount sufficient to deliver to a patient a therapeutically effective amount for the desired indication, without causing serious toxic effects in the patient treated.

In certain embodiments, the active compound is conveniently administered in any suitable unit dosage form, including but not limited to one containing 1 to 3000 mg, preferably 5 to 500 mg of active ingredient per unit dosage form. An oral dosage of 10-250 mg is usually convenient.

The actual dosage amount of a composition of the subject technology administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

in certain embodiments, pharmaceutical compositions may comprise, for example, at least about 0.1% of an active compound, i.e., at least one of the compounds of Formula (I), (II), (III) or a pharmaceutically acceptable salt, solvate, hydrate, prodrug or metabolite thereof. In other embodiments, the active compound may comprise between about 1% to about 75% of the weight of the unit, or between about 5% to about 50%, for example, and any range derivable therein. In other non-limiting examples, a dose may also comprise about 0.001 microgram/kg/body weight to about 5 microgram/kg/body weight, or about 1 microgram/kg/body weight to about 50 microgram/kg/body weight, or about 20 milligram/kg/body weight to about 150 milligram/kg/body weight, or about 100 milligram/kg/body weight to about 300 milligram/kg/body weight, or about 200 milligram/kg/body weight to about 1000 milligram/kg/body weight or more per administration, and any range derivable therein. In non-limiting examples of a derivable range from the numbers listed herein, a range of about 1 microgram /kg/body weight to about 50 milligram/kg/body weight, or from about 20 microgram/kg/body weight to about 500 milligram/kg/body weight, etc., can be administered.

Route of Administration

In accordance with the methods of the subject technology, the described arylquinoline, arylquinolone and arylthioquinolone derivatives of the subject technology or a pharmaceutically acceptable salt, solvate, hydrate or prodrug thereof, may be administered to a subject in a variety of forms depending on the selected route of administration, as will be understood by those skilled in the art. The active compound of the disclosure may be administered, for example, by oral, parenteral (including subcutaneous, intradermal, intramuscular, intravenous, intraperitoneal, intratumoral and intraarticular, intratumoral, transepithelial, nasal, intrapulmonary, intrathecal), buccal, sublingual, nasal, rectal, topical (including dermal, buccal, sublingual and intraocular), or transdermal administration as well as those for administration by inhalation. Parenteral administration includes intravenous, intraperitoneal, subcutaneous, intramuscular, intratumoral, transepithelial, nasal, intrapulmonary, intrathecal, rectal and topical modes of administration. Parenteral administration may be by continuous infusion over a selected period of time

Alternatively, the compounds of this disclosure may be incorporated into formulations for any route of administration including for example, oral, topical and parenteral including intravenous, intramuscular, eye or ocular, intraperitoneal, intrabuccal, transdermal and in suppository form. Of course, one of ordinary skill in the art may modify the formulations within the teachings of the specification to provide numerous formulations for a particular route of administration without rendering the pharmaceutical compositions unstable or compromising their therapeutic activity. It is also well within the routineer's skill to modify the route of administration and dosage regimen of a particular compound in order to manage the pharmacokinetics of the present compounds for maximum beneficial effect to the patient.

Methods of Treatment

In an embodiment, the subject technology is directed to methods for treating cancer or treating and/or inhibiting cancer metastasis in a subject comprising administering to the subject an effective amount of a compound or composition of one or more compounds of Formula (I), (II), and/or Formula (III) and/or one or more pharmaceutically acceptable salt, solvate, hydrate, prodrug or metabolite thereof.

For example, the subject technology contemplates methods of treating various cancers and complications thereof. More particularly, the subject technology relates to methods for inhibiting the growth of benign and malignant cancer, including a malignant tumor or cancer comprising exposing the tumor to an inhibitory or therapeutically effective amount or concentration of at least one arylquinoline, arylquinolone or arylthioquinolone derivative or pharmaceutically acceptable salts or pharmaceutically acceptable composition thereof. Treatment of internal malignancies such as eye or ocular cancer, rectal cancer, colon cancer, cervical cancer, prostate cancer, breast cancer, liver cancer and bladder cancer, and age-related cancer among numerous others are contemplated by the subject technology.

Accordingly, the compounds and/or compositions of the subject technology are useful for treating animals, and in particular, mammals, including humans, as patients. Thus, humans and other animals, and in particular, mammals, suffering from cancer can be treated by administering to the patient an effective amount of one or more of the arylquinoline, arylquinolone and arylthioquinolone derivatives according to the subject technology, or its derivative or a pharmaceutically acceptable salt thereof, optionally in a pharmaceutically acceptable carrier or diluent, either alone, or in combination with other known pharmaceutical agents (depending upon the disease to be treated). Treatment according to the subject technology can also be by administration of the compounds and/or compositions of the subject technology in conjunction with other conventional cancer therapies, such as radiation treatment or surgery or administration of other anti-cancer agents.

In certain embodiments, the subject technology can find application in the treatment of any disease for which delivery of a therapeutic arylquinoline, arylquinolone and arylthioquinolone derivative or a pharmaceutically acceptable salt, solvate, hydrate, prodrug or metabolite thereof to a cell or tissue of a subject is believed to be of therapeutic benefit. Examples of such diseases include hyperproliferative diseases and quiescent malignant diseases. In particular embodiments, the disease is a hyperproliferative disease, such as cancer of solid tissues or blood cells. Quiescent malignant diseases that can be treated by an arylquinoline, arylquinolone and arylthioquinolone derivative of the subject technology or a pharmaceutically acceptable salt, solvate, hydrate, prodrug or metabolite thereof include, for example, chronic lymphocytic leukemia.

For example, a compound or composition of an arylquinoline, arylquinolone and arylthioquinolone derivative of the subject technology or a pharmaceutically acceptable salt, solvate, hydrate, prodrug or metabolite thereof can be administered to treat a hyperproliferative disease. The hyperproliferative disease may be cancer, leiomyomas, adenomas, lipomas, heinangiomas, fibromas, pre-neoplastic lesions (such as adenomatous hyperplasia and prostatic intraepithelial neoplasia), carcinoma in situ, oral hairy leukoplakia, or psoriasis, for example.

The cancer may be a solid tumor, metastatic cancer, or non-metastatic cancer. in certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. In certain embodiments, the cancer is ovarian cancer. In particular aspects, the cancer may be a chemo-resistant cancer, i.e., refractive forms of cancer, such as taxane-resistant or cisplatin resistant cancer.

In another aspect, the subject technology provides a method for promoting secretion of Par-4 in a cell by contacting the cell with an effective amount of an arylquinoline derivative of the subject technology. In another aspect, there is provided a method for promoting apoptosis in a cancer cell by contacting the cell with an effective amount of an arylquinoline derivative of the subject technology.

Combination Therapy

The active compounds of the subject technology, i.e., one or more compounds of Formula (I) to (III) and/or one or more pharmaceutically acceptable salt, solvate, hydrate, prodrug or metabolite thereof can also be mixed with other active materials that do not impair the desired action, or with materials that supplement the desired action, such as chemotherapeutic agents, NF-κB inhibitors, other anticancer agents, and in certain instances depending upon the desired therapy or target, antibiotics, antifungals, antinflammatories, antiviral compounds or other agents having a distinct pharmacological effect.

The methods and compositions of the subject technology further provide combination therapies which can enhance the therapeutic or protective effect of the compounds of the subject technology, and/or increase the therapeutic effect of another anti-cancer. Therapeutic and prophylactic methods and compositions can be provided in a combined amount effective to achieve the desired effect, such as the killing of a cancer cell and/or the inhibition of cancer metastasis. This process may involve contacting the cells with, for example, a therapeutic nucleic acid, such as a chemotherapeutic agent or an inhibitor of gene expression, as a second therapy. A tissue, tumor, or cell can be contacted with the compounds or compositions of the subject technology and one or more additional anti-cancer treatment. For example, an additional anticancer treatment may include a chemotherapeutic agent, an NF-κB inhibitor, an anti-hormonal agent, radiotherapy, surgical therapy, or immunotherapy.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBl-TMl); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin garnmall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomyrin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carrnofur, cytarabine, dideoxyuridine, doxifluridine, enocitabine, floxuridine; androgens such as calusterone, dromostanolone propionate, epitiostanol, inepitiostane, testolactone; anti-adrenals such as aminoglutethimide, mitotane, trilostane; folic acid replenisher such as trolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanrnol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-Il); topoisomerase inhibitor RFS 2000; difluorometihylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristine, vinblastine and methotrexate and pharmaceutically acceptable salts, acids or derivatives of any of the above.

Examples of nf-κb inhibitors include 9-methylstreptimidone, n-stearoyl phytosphingosine, 2-(1,8-naphthyridin-2-yl)-phenol, 5-aminosalicylic acid, cape (caffeic acid phenethylester), diethylmaleate, ethyl 3,4-dihydroxycinnamate, helenalin, nf-κb activation inhibitor ii, nfκb activation inhibitor iii, glucocorticoid receptor modulator, cpda, aspirin, ppm-18, pyrrolidinedithiocarbamic acid ammonium salt, rocaglamide, sodium salicylate, andrographolide, (±)4-hydroxynon-2-enal, ps-1145 dihydrochloride, pioglitazone, sulindac sulfide, isohelenin, diethyldithiocarbamic acid sodium salt trihydrate, curcumin (synthetic), trichodion (which can be purchased, e.g., from Santa Cruz Biothechnology, Inc. Dallas, Tex.).

Also included in the formulations may be anti-hormonal agents that act to regulate or inhibit hormone action on tumors such as anti-estrogens and selective estrogen receptor modulators (SERMs), including, for example, tamoxifen, raloxifene, droloxifene, A-hydroxytamoxifen, trioxifene, keoxifene, LYI 17018, onapristone, and toremifene; aromatase inhibitors that inhibit the enzyme aromatase, which regulates estrogen production in the adrenal glands, such as, for example, 4(5)-imidazoles, aminoglutethimide, niegestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analogue); antisense oligonucleotides, particularly those which inhibit expression of genes in signaling pathways implicated in abherant cell proliferation, such as, for example, PKC-alpha, Raft and H-Ras; ribozymes such as a VEGF expression inhibitor and a HER2 expression inhibitor; vaccines such as gene therapy vaccines and pharmaceutically acceptable salts, acids or derivatives of any of the above.

In an embodiment, a therapeutic formulation or composition set forth herein, which comprises one or more compounds of Formula (I) to Formula (III) and/or one or more pharmaceutically acceptable salt, solvate, hydrate, prodrug or metabolite thereof, may be administered before, during, after or in various combinations relative to a second anti-cancer treatment. The administrations may be in intervals ranging from concurrently to minutes to days to weeks. In embodiments where the arylquinoline derivative containing composition is provided to a patient separately from an additional anti-cancer agent, one would generally ensure that a significant period of time did not expire between the time of each delivery, such that the two agents would still be able to exert an advantageously combined effect on the patient. In such instances, it is contemplated that one may provide a patient with the inhibitor of gene expression therapy and the anti-cancer therapy within about 12 to 24 or 72 h of each other and, more preferably, within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations.

Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Administration of any compound or therapy of the subject technology to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as radiation and surgical intervention, may be applied in combination with the described therapy.

In specific aspects, it is contemplated that a standard therapy will include chemotherapy, radiotherapy, immunotherapy, surgical therapy or gene therapy and may be employed in combination with the combination therapy described herein.

Articles of Manufacture

In another embodiment of the disclosure, an article of manufacture, or “kit”, containing materials useful for the treatment of the diseases and disorders described above is provided. In one embodiment, the kit comprises a container comprising at least one compound of Formula (I)-(III), and/or one or more pharmaceutically acceptable salt, solvate, hydrate, prodrug or metabolite thereof.

The kit may further comprise a label or package-insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula (I)-(III) or a formulation thereof which is effective for treating the condition and may have a sterile access port (for example, the container may he an intravenous solution bag or a vial having a stopper pierceable by a hypodermic injection needle). At least one active agent in the composition is a compound of Formula (I)-(III). The label or package insert indicates that the composition is used for treating the condition of choice, such as cancer.

In an embodiment, the kit includes two separate pharmaceutical compositions: one containing a compound of the subject technology, and a second pharmaceutical compound. In another embodiment, an assay or diagnostic kit includes a labeled compound of the subject technology and one or more reagents necessary for detecting the labeled compound upon binding to its target in-vivo or in-vitro. In a related embodiment, the kit includes a package insert that describes the steps necessary for carrying out the detection assay.

In another embodiment, a kit of the disclosure further comprises a needle or syringe, preferably packaged in sterile form, for injecting the composition, and/or a packaged alcohol pad. Instructions are optionally included for administration of arylquinoline derivatives by a clinician or by the patient.

Diagnostic Methods and Diagnostic Probes

Another aspect of the subject technology provides compounds having general formulas (I)-(III) with a linker moiety (hydrophobic linkers, hydrophilic linkers, photo-cleavable linkers, redox reaction-cleavable linkers), wherein the linker moiety is covalently bonded to a label molecule (a label could be a fluorophor, biotin, different polymer beads and different reactive groups).

The compounds of the subject technology when biotinylated provide suitable means for non-radioactive detection of target molecules that may play a role in apoptosis induction in cells. Therefore, another aspect of the subject technology relates to the use of biotinylated arylquinoline derivatives as a diagnostic reagent for detecting or monitoring the presence or levels of vimentin in a complex protein sample. A complex protein sample contains multiple proteins, and may additionally contain other contaminants. Non-limiting examples of a complex protein sample include tumor tissues, biopsy, serum and cell extracts.

In one embodiment, the subject technology relates to a method of detecting, monitoring or analyzing the levels of vimentin in a complex protein sample, said method comprising adding a labeled compound of Formula (I)-(III) to said complex protein mixture under conditions whereby said labeled compound covalently conjugates to vimentin; isolating. the conjugated vimentin by a suitable affinity-based separation method, removing unbound proteins, detecting the level of vimentin following the separation. In a related embodiment, the detection can be accomplished by measuring a fluorescence signal emitted from the compound of Formula (I)-(III). In another related embodiment, the detection can be accomplished by measuring a fluorescence signal emitted from a label bound via a linker to the compound of Formula (I)-(III). The detection step can also be accomplished using various analytical procedures that known to the artisan for separating and analyzing complex protein mixtures. These analytical procedures include chromatographic methods such as HPLC, FPLC, ion exchange, size exclusion, mass spectrometry, and the like.

The linker moiety that can be used to attach a detectable label to the compounds of the subject technology can be any appropriate linker. For example, the linker moiety comprises a repeating alkyleneoxy structure (polyethylene glycols, or “PEG”). Thus, one of skill in the art can select the linker moiety of the compounds of the subject technology in order to provide additional specificity of them for vimentin.

In an embodiment, it is desirable to have a detectable label associated with a compound of the subject technology to allow the compound-vimentin complex to be captured and washed free of other components of the reaction mixture. The label will generally be under about 1 kDa. Biotin is a conventional label or ligand, particularly analogs such as dethiobiotin and deiminobiotin, which can be readily displaced from streptavidin by biotin. However, any small molecule will suffice that can be captured and released under convenient conditions.

Affinity purification of biological molecules, for example proteins, is known in the art and allows the purification of molecules by exploiting the binding affinity of the target molecule for a molecular binding partner. Examples of affinity purification methods are fusion tag protein purification, avidin-biotin system, pull-down assay and the like.

Drug Screening Assays

In another aspect, the subject technology provides assays for screening test compounds that interfere with vimentin binding to PAR-4 or which cause release of vimentin-bound PAR-4. In a typical assay, cells, such as HEL cells, are seeded onto an appropriate support, such as 60-mm plates containing supplemented growth medium (e.g., supplemented with 0.1% serum) at a desired confluency, such as 40-70% confluency, and treated with the test compound (e.g., 500 nM) or control vehicle. The cells are allowed to grow for a period of time, such as 24 hours, after which the conditioned medium (CM) is concentrated (e.g., concentrated 30× to 100 μl using Millipore tubes (Amicon Ultra centrifugal filters, Ultracel-10K) by 15 min centrifugation at 4500 rpm). The concentrated CM is then subjected to co-immunoprecipitation using detectably labeled PAR-4 and vimentin antibodies in the presence of various concentrations of purified PAR-4 and the precipitated proteins are resolved by SDS-PAGE and analyzed by Western blot analysis. The amount of immunoprecipitated PAR-4-vimentin complex is compared to that of control samples, For example, chemiluminescent signals may be quantified in the BIO-RAD Molecular Imager ChemiDoc XRS±Imaging system using Quantity One software. Induction of Par-4 secretion by the test drug (e.g., 500 nM), as judged by this Western blot analysis procedure, will indicate dissociation of Par-4 from vimentin. Additionally, experiments can be undertaken to to determine whether the test compound disrupts the interaction between vimentin and Par-4 using any test drug that shows elevated secretion of Par-4 by Western blot analysis or other such assay. For such experiments, cells may be treated with the test drug (e.g., 500 nM) or vehicle for an appropriate period of time, such as 24 hours, and then cell lysates are prepared and subjected to immunoprecipitation using an appropriate amount of a Par-4 antibody, vimentin antibody or control IgG antibody by using any standard irnmunoprecipitation procedure. Following immunosuppression, the complexes are resolved, preferably by Western blot analysis as described above. If the drug inhibits the binding of Par-4 to vimentin, it will be reflected in the lack of co-immunoprecipitation of Par-4 with the vimentin antibody, and co-immunoprecipitation of vimentin with the Par-4 antibody.

### EXAMPLES

The following examples are intended to further illustrate certain preferred embodiments of the disclosure and are not limiting in nature. Those skilled in the art will recognize, or be able to ascertain, using no more than routine experimentation, numerous equivalents to the specific substances and procedures described herein.

Chemicals and Instruments. Chemicals were purchased from Sigma Aldrich or Fisher Scientific, or were synthesized according to literature procedures. Solvents were used from commercial vendors without further purification unless otherwise noted. Nuclear magnetic resonance spectra were determined in dimethyl sulfoxide-d₆ using a Varian instrument (¹H, 400 MHz; ¹³C, 100 Mz). High resolution electrospray ionization mass spectra (HRMS ESI) were recorded on a LTQ-Orbitrap Velos mass spectrometer (Thermo Fisher Scientific, Waltham, Mass., USA), The FT resolution was set at 100,000 (at 400 m/z), Samples were introduced through direct infusion using a syringe pump with a flow rate of 5 μL/min. Purity of compounds was >95% as established by combustion analyses that was conducted by Atlantic Microlabs (Norcross, Ga.). Compounds were chromatographed on preparative layer Merck silica gel F254 purchased from Fisher Scientific (Pittsburgh, Pa.) unless otherwise indicated. Organic solutions were dried over anhydrous magnesium sulfate unless otherwise specified.

General Procedure for the Synthesis of Arylquinolines (1). To a solution of 2.38 mmol (1.3 equiv) of an appropriate phenylacetonitrile in 3 mL of anhydrous DMF at 0° C. was added 2.38 mmol (1.3 equiv) of potassium tert-butoxide. The mixture was stirred for 15 min, and 1.83 mmol of 2-aminoaldehyde 5 in 1 mL of anhydrous DMF was added dropwise at 0° C. The mixture was allowed to warm to the room temperature and stirred for 3 h at 90° C. After cooling, the mixture was quenched in water with vigorous stirring. The solution was adjusted to pH 7 only in the case of 2-amino-3-(2-fluorophenyl)quinoline-7-carboxylic acid. A precipitate was collected by filtration and purified by recrystallization and/or chromatography as noted for individual compounds described below.

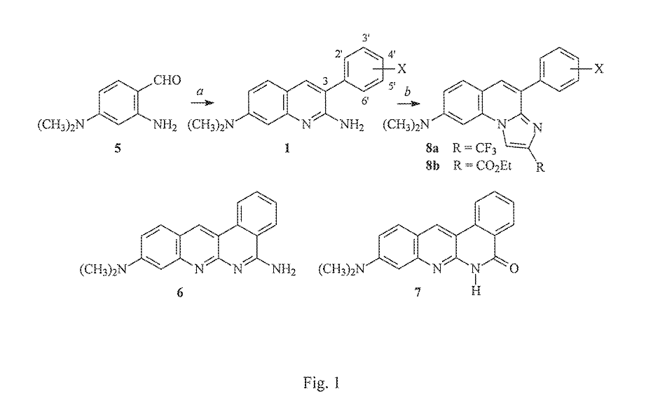

Synthesis of 3-Arylquinolines with Variations in the 3-Aryl Substituent. Synthesis of 3-arylquinolines 1 utilized a Friedlander condensation¹⁵ of 2-amino-4-(N,N-dimethylamino)benzaldehyde (5) with various arylacetonitriles (FIG. 1) to afford an array of halogenated arylquins 1 (Table 1) that possessed Par-4 secretory activity (vide infra). The Friedlander synthesis of arylquins with other electron-withdrawing substituents, such as an ortho-cyano or ortho-carboxylate group, produced the biologically inactive dibenzo[b,f][1,8]naphthyridine 6 or dibenzo[b,f][1,8]naphthyridin-5(6H)-one¹⁶ (7), respectively (FIG. 1). In addition to the tetracyclic system in 6 and 7, condensation of 1a with 3-chloro-1,1,1-trifluoroacetone or ethyl 3-chloropyruvate furnished another tetracyclic system, the imidazo[1,2-a]quinolines¹⁷ 8a and 8b, respectively (FIG. 1). The use of still other arylacetonitriles afforded arylquins with electron-donating hydroxy, methoxy or methyl substituents or with halogenation patterns different from those in Table 1, but these analogs lacked sufficient activity to warrant further interest.

TABLE 1
SAR study of modifications of the 3-aryl group in arylquinolines 1.

| | | | | | | Relative Levels | | |
|---|---|---|---|---|---|---|---|---|
| | | | | | | of Par-4 | Ratio of Par-4 | |
| | | | | | | Secretion by | Secretion by | |
| | | | | | | Arylquins | Arylquin 1 | |
| | | | | | | Administered at | Relative to | hERG IC₅₀ |
| Arylquin | C-2′ | C-3′ | C-4′ | C-5′ | C-6′ | 500 nM | Vehicle | (μM) |
| 1a⁸ | F | H | H | H | H | 104.7 ± 10.7 | 3.5 | 3.74 ± 1.17 |
| 1b | Cl | H | H | H | H | 88.7 ± 8.4 | 3.0 | 7.70 ± 2.07 |
| 1c | Br | H | H | H | H | 33.6 ± 14.8 | | 5.69 ± 2.56 |
| 1d⁸ | H | F | H | H | H | 66.8 ± 10.9 | | 1.59 ± 0.39 |
| 1e | H | Cl | H | H | H | 78.3 ± 15.3 | 2.6 | 2.50 ± 0.59 |
| 1f⁸ | H | H | F | H | H | 12.3 ± 2.0 | | 30.2 ± 10.3 |
| 1g | H | H | Cl | H | H | 21.3 ± 4.6 | | 8.19 ± 1.13 |
| 1h | F | F | H | H | H | 93.2 ± 10.6 | 3.1 | 5.31 ± 1.05 |
| 1i | Cl | Cl | H | H | H | 88.4 ± 1.3 | 3.0 | 5.51 ± 0.67 |
| 1j | F | H | F | H | H | 80.8 ± 3.0 | 2.7 | 1.44 ± 0.04 |
| 1k | F | H | H | F | H | 86.2 ± 7.5 | 2.9 | 4.85 ± 1.11 |
| 1l | Cl | H | H | Cl | H | 64.9 ± 13.1 | 2.2 | 6.91 ± 0.50 |
| 1m | F | H | H | H | F | 44.6 ± 4.6 | 1.5 | 3.32 ± 1.91 |
| 1n | H | F | F | H | H | 58.4 ± 14.1 | 2.0 | 2.79 ± 0.97 |
| 1o | H | F | H | F | H | 47.4 ± 16.8 | | 4.10 ± 0.49 |

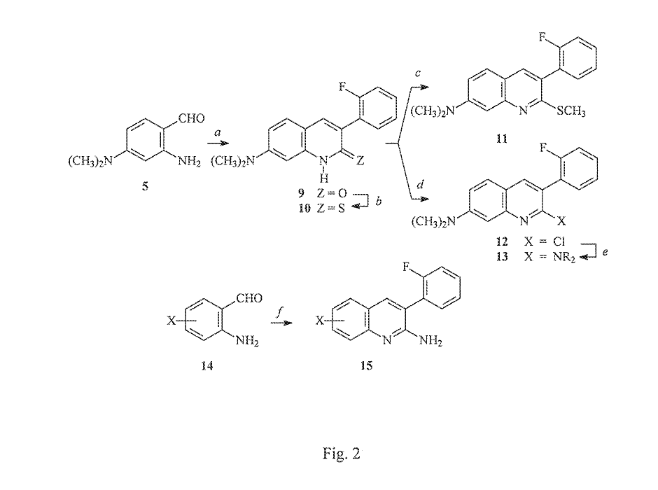

Synthesis of 3-Arylquinolines with Variations in the Quinoline Ring. Synthesis of arylquinolines modified at the C-2 position again utilized the Friedlander condensation¹⁵ of 2-amino-4-(N,N-dimethylamino)benzaldehyde (5) with 2-(2′-fluorophenypacetyl chloride to afford the quinolone 9 that was elaborated to the thioquinolone 10, 2-thiomethoxyquinoline 11, 2-chloroquinoline 12, and 2-dialkylaminoquinoline 13 in straightforward reactions (FIG. 2). Modifications at the C-5, C-6 and C-7 positions also utilized Friedlander condensation^{s} of appropriately substituted aminobenzaldehydes 14 with 2-(2′-fluorophenypacetonitrile to afford arylquins 15 (FIG. 2 and Table 2).

TABLE 2
SAR study of modifications of quinoline rings in arylquins.

| | | | | | Relative Levels | | |
|---|---|---|---|---|---|---|---|
| | | | | | of Par-4 | Ratio of Par-4 | |
| | | | | | Secretion by | Secretion by | |
| | | | | | Arylquins | Arylquin 1 | |
| | | | | | Administered at | Relative to | hERG IC₅₀ |
| Arylquin | C-2 | C-5 | C-6 | C-7 | 500 nM | Vehicle | (mM) |
| 1a⁸ | NH₂ | H | H | N(CH₃)₂ | 104.7 ± 10.7 | 3.5 | 3.74 ± 1.17 |
| 9 | quinolone | H | H | N(CH₃)₂ | 33.2 ± 0.9 | 1.1 | >100 |
| 10 | thioquinolone | H | H | N(CH₃)₂ | 51.7 ± 4.9 | 1.7 | >100 |
| 11 | SCH₃ | H | H | N(CH₃)₂ | 75.4 ± 7.0 | 2.5 | ca. 100 |
| 12 | Cl | H | H | N(CH₃)₂ | 104.5 ± 26.9 | 3.5 | ca. 100 |
| 13a | morpholino | H | H | N(CH₃)₂ | 54.4 ± 2.8 | 1.8 | 23.3 ± 3.05 |
| 13b | N-methylpiperazinyl | H | H | N(CH₃)₂ | 65.6 ± 3.6 | 2.2 | 9.41 ± 1.89 |
| 15a⁸ | NH₂ | H | H | H | 82.1 ± 21.2 | 2.8 | 14.6 ± 5.82 |
| 15b | NH₂ | F | H | H | 83.4 | 2.8 | |
| 15c | NH₂ | Cl | H | H | 63.2 ± 20.5 | 2.1 | ca. 100 |
| 15d | NH₂ | Br | H | H | 44.6 | 1.5 | |
| 15e | NH₂ | H | Cl | H | 81.9 ± 11.2 | 2.8 | 12.7 ± 6.83 |
| 15f | NH₂ | H | H | Cl | <50 | | |
| 15g | NH₂ | H | H | OCH₃ | <50 | | |
| 15h | NH₂ | H | H | CO₂H | <50 | | |

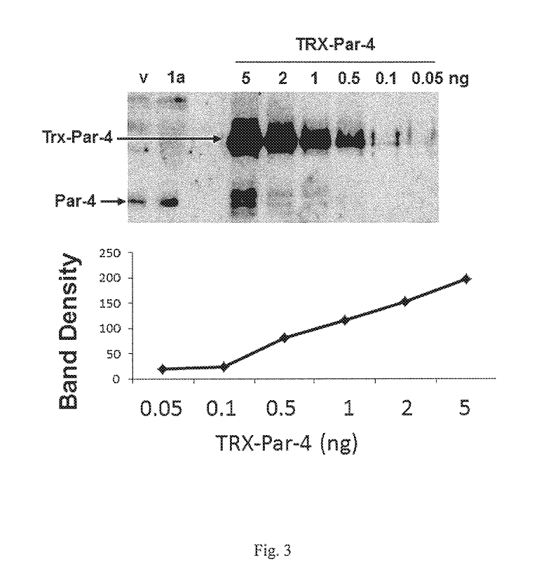

Biological activity of 3-Arylquinolines. Prior studies indicated that arylquinoline 1a induced the robust secretion of Par-4 from normal cells, and the amount of Par-4 secreted by 1a in cell culture-conditioned medium (CM) was adequate to induce apoptosis of diverse lung and prostate cancer cells.⁸ To determine if variations in the substituents in the 3-aryl group (Table 1) or the quinoline ring (Table 2) enhanced the secretory capability of these arylquins relative to arylquinoline 1a, we treated mouse fibroblasts with these arylquinolines, arylquincolones or arylthioquinolones or with 1a at 500 nM concentrations using vehicle alone as a control. After 24 h exposure, the CM and lysates were subjected to quantitative Western blot analysis for Par-4 and collagen (CollA1). CollA1 secretion from the cells was not affected by exogenous application of arylquins, and therefore, CollA1 served as a loading control to normalize Par-4 secretion in the CM, as described in prior work.⁸ Quantitation of Par-5 secretion (FIG. 3) using arylquinoline 1a showed an two-fold increase in Par-4 concentrations in the CM from basal levels of 280 pM (vehicle alone) to 540 pM (arylquinoline 1a).

3-(2-Chlorophenyl)-N⁷,N⁷-dimethylquinoline-2,7-diamine (1b). Yield 86%, mp 147-148° C. (from 2-propanol).

3-(2-Bromophenyl)-N⁷,N⁷-dimethylquinoline-2,7-diamine (1c). Yield 61%, mp 159-160° C. (from acetonitrile).

3-(3-Chlorophenyl)-N⁷,N⁷-dimethylquinoline-2,7-diamine (1e). Yield 85%, mp 144-145° C. (from acetonitrile).

3-(4-Chlorophenyl)-N⁷,N⁷-dimethylquinoline-2,7-diamine (1g). Yield 81%, mp 169-170° C. (from acetonitrile).

3-(2,3-Difluorophenyl)-N⁷,N⁷-dimethylquinoline-2,7-diamine (1h). Yield 76%, R_{f}=0.55 (methanol-dichloromethane 1:10), mp 169-171° C.

3-(2,3-Difluorophenyl)-N⁷,N⁷-dimethylquinoline-2,7-diamine (1i). Yield 72% (from acetonitrile), mp 214-215° C.

3-(2,4-Difluorophenyl)-N⁷,N⁷-dimethylquinoline-2,7-diamine (1j). Yield 53% R_{f}=0.59 (methanol-dichloromethane 1:10), mp 174-175° C.

3-(2,5-Difluorophenyl)-N⁷,N⁷-dimethylquinoline-2,7-diamine (1k). Yield 85%, (methanol-dichloromethane 1:10), mp 165-166° C.

3-(2,5-Difluorophenyl)-N⁷,N⁷-dimethylquinoline-2,7-diamine (1l). Yield 46% (from acetonitrile), mp 164-166° C.

3-(2,6-Difluorophenyl)-N⁷,N⁷-dimethylquinoline-2,7-diamine (1m). Yield 36%, R_{f}=0.41 (methanol-dichloromethane 1:10), mp 148-150° C.

3-(3,4-Difluorophenyl)-N⁷,N⁷-dimethylquinoline-2,7-diamine (1n). Yield 91%, R_{f}=0.53 (methanol-dichloromethane 1:10, and then recrystallized from acetonitrile), mp 163-164° C.

3-(3,5-Difluorophenyl)-N⁷,N⁷-dimethylquinoline-2,7-diamine (1o). Yield 80%, R_{f}=0.52 (methanol-dichloromethane 1:10 and then recrystallized from ethanol), mp 160-162° C.

N⁹,N⁹-Dimethyldibenzo[b,f][1,8]naphthyridine-5,9-diamine (6), Yield 85%, mp >250° C. (from methanol-DMF). ¹H NMR: δ 9.18 (s, 1H), 8.68 (d, 1H, J=8.0 Hz), 8.35 (d, 1H, J=8.0 Hz), 7.86 (d, 1H, J=9.6 Hz), 7.83 (d, 1H, J=7.6 Hz), 7.63 (t, 1H, J=7.8 Hz), 7.45 (s, 2H), 7.22 (dd, 1H, J=9.4 Hz, 2.6 Hz), 6.87 (d, 1H, J=2.0 Hz), 3.09 (s, 6H). ¹³C NMR: δ 158.79, 155,13, 151.33, 150.68, 134.08, 131.00, 130.24, 128.76, 126.16, 124.66, 122.46, 118.55, 117.85, 114.80, 112.06, 104.16, 40.03 (2C). HRMS (ESI) Calcd for C₁₈H₁₇N₄ [MH+]: 289.1448. Found: 289.1447. Anal. Calcd for C₁₈H₁₆N₄: C, 74.98; H, 5.59. Found: C, 74.70; H, 5.69.

9-(Dimethylamino)dibenzo[b,f][1,8]naphthyridin-5(6H)-one (7). Yield 95%, mp >250° C. (from ethanol-DMF). ¹H NMR: δ 11.80 (s, 1H), 9.15 (s, 1H), 8.52 (d, 1H, J=8.0 Hz), 8.28 (d, 1H, J=7.2 Hz), 7.87 (d, 1H, J=7.2 Hz), 7.86 (d, 1H, J=9.6 Hz), 7.61 (t, 1H, J=7.6 Hz), 7.24 (dd, 1H, J=9.0 Hz, 2.6 Hz), 6.81 (d, 1H, J=2.4 Hz), 3.10 (s, 6H). ¹³C NMR: δ 162.31, 152,02, 149.04, 148.19, 133.69, 133.07, 131.51, 129.26, 127.62, 127.61, 124.76, 122.39, 117.72, 114.92, 109.98, 103.28, 39.73 (2C). HRMS (ESI) Calcd for C₁₈H₁₆N₃O [MH+]: 290.1288. Found: 290.1286. Anal. Calcd for C₁₈H₁₅N₃O: C, 74.72; H, 5.23. Found: C, 74.49; H, 5.05.

3-(2-Fluorophenyl)-N,N-dimethyl-2-(methylthio)quinolin-7-amine (11). To a solution of 200 mg (0.67 mmol) of 10 in 3 mL of DMF were added 111 mg (0.80 mmol, 1.2 equiv) of K₂CO₃ and 95 mg (0.67 mmol, 1 equiv) of iodomethane. The mixture was stirred for 12 h at room temperature, poured into water, extracted with dichloromethane, dried over anhydrous MgSO₄ and concentrated under reduced pressure. The crude residue was purified by chromatography using 1:10 methanol-dichloromethane (R_{f}=0.74) to afford 120 mg (57%) of 11 as a pale yellow solid: mp 161-162° C. (from ethanol). ¹ H NMR: δ 7.85 (s, 1H), 7.71 (d, 1H, J=9.21 Hz), 7.53-7.47 (m, 1H), 7.42 (td, 1H, J=7.2 Hz, 2.0 Hz), 7.35-7.28 (m, 2H), 7.18 (dd, 1H, J=8.8 Hz, 2.6 Hz), 6.93 (d, 1H, J=2.4 Hz), 3.08 (s, 6H), 2.53 (s, 3H). ¹³C NMR: δ 159.66 (d, J=243.8 Hz), 157.95, 151.56, 148.86, 135.54, 132.31 (d, J=2.7 Hz), 130.51 (d, J=8.1 Hz), 128.51, 125.31 (d, J=16.1Hz), 124.45 (d, J=3.3 Hz), 123.20, 117.16, 115.65 (d, J=22.2 Hz), 114.88, 104.87, 40.02 (2C), 12.72. HRMS (ESI) Calcd for C₁₈H₁₈FN₂S [MH+]: 313.11692, Found: 313.11790. Anal. Calcd for C₁₈H₁₇FN₂S: C, 69.20; H, 5.48. Found: C, 68.93; H, 5.66.

2-Chloro-3-(2-fluorophenyl)-N,N-dimethylquinolin-7-amine (12). A mixture of 500 mg (1.77 mmol, 1 equiv) of 9 and 5 mL of oxyphosphorous trichloride was refluxed for 3 h. After cooling, the mixture was poured on ice and neutralized with 10% aq. Na₂CO₃. A precipitate was collected by filtration, dried in vacuum and purified by chromatography using 1:5 ethylacetate-hexane (R_{f}=0.25) to afford 416 mg (78%) of 12 as a pale yellow solid: mp 170-172° C. ¹H NMR: δ 8.20 (s, 1H), 7.83 (d, 1H, J=9.2 Hz), 7.54-7.46 (m, 2H), 7.36-7.29 (m, 3H), 6.95 (d, 1H, J=2.4 Hz), 3.09 (s, 6H). ¹³C NMR: δ 159.36 (d, J=243.6 Hz), 151.97, 148.83, 148.76, 139.50, 132.05 (d, J-3.0Hz), 130.50 (d, J=8.4Hz), 128.59, 125,54 (d, J-16,0Hz), 125.51 (d, J=3.8 Hz), 122.92, 118.79, 116.87, 115.52 (d, J=21.2 Hz), 104.18, 39.86 (2C). HRMS (ESI) Calcd for C₁₇H₁₄ClFN₂ [MH+]: 301.09023. Found: 301.08961. Anal. Calcd for C₁₇H₁₄ClFN₂: C, 67.89; H, 4.69. Found: C, 67.76; H, 4.52.

3-(2-Fluorophenyl)-NA-dimethyl-2-morpholinoquinolin-7-amine (13a). The procedure described for the preparation of 13b was repeated using 100 mg (0.33 minol) of 12 and 145 mL (1.66 mmol, 5 equiv) of morpholine. The product was chromatographed using 1:20 methanol-dichloromethane (R_{f}=0.60) to afford 52 mg (45%) of 13a: mp 166-168° C. ¹H NMR: δ 7.87 (s, 1H), 7.64 (d, 1H, J=9.2 Hz), 7.60 (td, 1H, J=8.0 Hz, 1.6 Hz), 7.45-7.40 (m, 1H), 7.34-7.28 (m, 2H), 7.05 (dd, 1H, J=8.6 Hz, 2.6 Hz), 6.79 (d, 1H, J=2.0 Hz), 3.48 (t, 4H, J=4.6 Hz), 3.04 (t, 4H, J=4.6 Hz), 3.05 (s, 6H). ¹³C NMR: δ 158.76 (d, J=244.4 Hz), 158.49, 151.51, 147.96, 139.23, 130.98 (d, J=12.0 Hz), 129.42 (d, J=8.4 Hz), 128.14, 127.26 (d, J=14.4 Hz), 124.68 (d, J=3.8 Hz), 117.15, 116.56, 115.97 (d, J=22.0 Hz), 113.49, 104.90, 65.87 (C), 49.21 (2C), 40.04 (2C). HRMS (ESI) Calcd for C₂₁H₂₃FN₃O [MH+]: 352.1820. Found: 352.1820. Anal. Calcd for C₂₁H₂₂FN₃O: C, 71.77; H, 6.31. Found: C, 71.59; H, 6.25.

3-(2-Fluorophenyl)-N,N-dimethyl-2-(4-methylpiperazin-1-yl)quinolin-7-amine (13b). A mixture of 100 mg (0.33 mmol) of 12 and 184 mL (1.66 mmol, 5 equiv) of 1-methylpiperazine was heated under reflux for 12 h. After cooling, the mixture was chromatographed using 1:10 methanol-dichloromethane (R_{f}=0.45) to afford 65 mg (54%) of 13b: mp 177-179° C. ¹H NMR: δ 7.83 (s, 1H), 7.62 (d, 1H, J=8.8 Hz), 7.56 (td, 1H, J=7.6 Hz, 1.4 Hz), 7.44-7.39 (m, 1H), 7.33-7.28 (m, 2H), 7.03 (dd, 1H, J=9.2 Hz, 2.8 Hz), 6.78 (d, 1H, J=2.0 Hz), 3.06 (t, 4H, J=4.6 Hz), 3.04 (s, 6H), 2.21 (t, 4H, J=4.6 Hz), 2.12 (s, 3H). ¹³C NMR: δ 158.75 (d, J=243.6 Hz), 158.66, 151.45, 148.00, 139.06, 130.97 (d, J=3.1 Hz), 129.27 (d, J=7.6 Hz), 128.08, 127.48 (d, J=14.4 Hz), 124.63 (d, J=3.1 Hz), 117.32, 116.47, 115.95 (d, J=22.1 Hz), 113.29, 104.93, 54.37 (2C), 48.42 (2C), 45.63, 40.06 (2C). FIRMS (ESI) Calcd for C₂₂H₂₆FN₄ [MH+]: 365.2136. Found: 365.2136. Anal. Calcd for C₂₂H₂₅FN₄: C, 72.50; H, 6.91. Found: C, 72.29; H, 6.68.

5-Fluoro-3-(2-fluorophenyl)quinolin-2-amine (15b). Yield 88%, mp 142-143° C. (from ethanol). ¹H NMR: δ 7.90 (s, 1H), 7.54-7.46 (m, 3H), 7.38-7.32 (m, 3H), 7.01-6.93 (m, 1H), 6.13 (s, 2H). ¹³C NMR: δ 159.54 (d, J=243.7 Hz), 157.86 (d, J=249.7 Hz), 156.58, 148.94 (d, J=3.8 Hz), 131.74 (d, J=3.0 Hz), 130.61 (d, J=8.4 Hz), 130.21 (d, J=4.6 Hz), 129.45 (d, J=9.9 Hz), 125.00 (d, J=3.1 Hz), 124.32 (d, J=16.0 Hz), 121.13 (d, J=3.0 Hz), 119.2.8 (d, J=2.3 Hz), 116.15 (d, J=21.2 Hz), 112.25 (d, J=15.2 Hz), 105,63 (d, J=19.7 Hz). HRMS (ESI) Calcd for C₁₅H₁₁F₂N₂ [MH+]: 257.0885. Found: 257.0889. Anal. Calcd for C₁₅H₁₀F₂N₂: C, 70.31; H, 3.93. Found: C, 70.08; H, 3.81.

5-Chloro-3-(2-fluorophenyl)quinolin-2-amine (15c). Yield 93%, mp 194-196° C. (from ethanol). ¹H NMR: δ 7.96 (s, 1H), 7.56-7.43 (m, 4H), 7.38-7.31 (m, 3H), 6.35 (s, 2H). ¹³C NMR: δ 159.51 (d, J=244.4 Hz), 156.45, 148.65, 133.58, 131.68 (d, J=3.1 Hz), 130.67 (d, J=8.3 Hz), 130.02, 129.72, 125.02 (d, J=3.0 Hz), 124.52, 124.21 (d, J=16.0 Hz), 121.69, 120.06, 120.00, 116.17 (d, J=21.3 Hz). HRMS (ESI) Calcd for C₁₅H₁₁ClFN₂ [MH+]: 273.0589. Found: 273.0588. Anal. Calcd for C₁₅H₁₀ClFN₂: C, 66.07; H, 3.70. Found: C, 66.01; H, 3.88.

5-Bromo-3-(2-fluorophenyl)quinolin-2-amine (15d). Yield 91%, mp 209-210° C. (from ethanol). ¹H NMR: δ 7.89 (s, 1H), 7.56-7.42 (m, 5H), 7.38-7.33 (m, 2H), 6.39 (s, 2H). ¹³C NMR: δ 159.51 (d, J=244.4 Hz), 156.52, 148.69, 136.17, 131.68 (d, J=3.1 Hz), 130.72 (d, J=8.4 Hz), 130.27, 125.26, 125.17, 125.06 (d, J=3.0 Hz), 129.15 (d, J=16.0 Hz), 121.38, 120.93, 120.38, 116.21 (d, J=21.3 Hz). HRMS (ESI) Calcd for C₁₅H₁₁BrFN₂ [MH+]: 317.0084. Found: 317.0086. Anal. Calcd for C₁₅H₁₀BrFN₂: C, 56.81; 3.18. Found: C, 56.63; H, 3.14.

6-Chloro-3-(2-fluorophenyl)quinolin-2-amine (15e). Yield 67%, R_{f}=0.28 (methanol-dichloromethane 1:50), mp 138-139° C. ¹ H NMR: δ 7.87 (s, 1H), 7.80 (d, 1H, J=1.6 Hz), 7.54-7.49 (m, 3H), 7.44 (td, 1H, J=7.4 Hz, 1.8 Hz) 7.37-7.32 (m, 2H), 6.20 (s, 2H). ¹³C NMR: δ 159.50 (d, J=244.4 Hz), 156.24, 146.18, 137.06, 131.68 (d, J=3.1 Hz), 130.55 (d, J=7.6 Hz), 129.70, 126.83, 126.24, 125,29, 124.96 (d, J=3.1 Hz), 124.35 (d, J=16.7 Hz), 123.50, 119.81, 116.14 (d, J=22.0 Hz). HRMS (ESI) Calcd for C₁₅H₁₁ClFN₂ [MH+]: 273.0589, Found: 273.0589, Anal. Calcd for C₁₅H₁₀ClFN₂: C, 66.07; H, 3.70. Found: C, 66.00; H, 3.82.

7-Chloro-3-(2-fluorophenyl)quinolin-2-amine (15f). Yield 80%, mp 178-180° C. (from isopropanol). ¹H NMR: δ 7.90 (s, 1H), 7.73 (d, 1H, J=8.8 Hz), 7.53-7.50 (m, 2H), 7.46 (td, 1H, J=7.6 Hz 1.6 Hz), 7.37-7.32 (m, 2H), 7.21 (td, 1H, J=8.6 Hz, 2.2 Hz), 6.27 (br s, 2H). ¹³C NMR: δ 159.52 (d, J=243.7 Hz), 156.72, 148.31, 137.70, 133.96, 131.70 (d, J=3.0 Hz), 130.50 (d, J=8.4 Hz), 129.47, 124.94 (d, J=3.8 Hz), 124.38 (d, J=16.7 Hz), 123.49, 121.83, 121.35, 119.17, 116.12 (d, J=21.3 Hz). HRMS (EST) Calcd for C₁₅H₁₁ClFN₂ [MH+]: 273.0589. Found: 273.0590. Anal. Calcd for C₁₅H₁₀ClFN₂: C, 66.07; H, 3.70. Found: C, 66.18;H, 3.76.

3-(2-Fluorophenyl)-7-methoxyquinolin-2-amine (15g). Yield 71%, R_{f}=0.58 (methanol-dichloromethane 1:2), mp 114-116° C. ¹H NMR: δ 7.78 (s, 1H), 7.59 (d, 1H, J=8.8 Hz), 7.51-7.46 (m, 1H), 7.45 (td, 1H, J=7.8 Hz, 1.8 Hz), 7.35-7.30 (m, 2H), 6.95 (d, 1H, J=2.4 Hz), 6.85 (dd, 1H, J=8.81 Hz, 2.8 Hz), 5.94 (s, 2H), 3.85 (s, 3H). ¹³C NMR: δ 160.63, 159.61 (d, J=243.7 Hz), 156.05, 149.16, 137.65, 131.85 (d, J=3.0 Hz), 130.12 (d. J=7.6 Hz), 128.76, 125.04, 124.87 (d, J=3.8 Hz), 117.58, 116.05 (d, J=22.0 Hz), 115.87, 113.25, 104.65, 55.12. HRMS (ESI) Calcd for C₁₆H₁₄FN₂O [MH+]: 269.1085. Found: 269.1085. Anal. Calcd for C₁₆H₁₃FN₂O: C, 71.63; H, 4.88. Found: C, 71.43; H, 4.99.

2-Amino-3-(2-fluorophenyl)quinoline-7-carboxylic add (15h). Yield 84%, mp >270° C. ¹H NMR: δ 13.13 (br s, 1H), 8.12 (s, 1H), 7.96 (s, 1H), 7.79 (d, 1H, J=8.4 Hz), 7.70 (dd, 1H, J=8.2 Hz, 1.8 Hz), 7.55-7.49 (m, 1H), 7.48 (td, 1H, J=7.4 Hz, 1.8 Hz), 7.38-7.33 (m, 2H), 6.32 (br s, 2H). ¹³C NMR: δ 167.60, 159.51 (d, J=244.4 Hz), 156.53, 146.81, 137.69, 131.67 (d, J=3.0 Hz), 131.59, 130.63 (d, J=8.3 Hz), 127.98, 126.46, 125.32, 124.98 (d, J=3.0 Hz), 124.29 (d, J=15.9 Hz), 121.18, 120.86, 116.15 (d, J=21.2 Hz), HRMS (ESI) Calcd for C₁₆H₁₂FN₂O₂ [MH+]: 283.0877. Found: 283.0875. Anal. Calcd for C₁₆H₁₁FN₂O₂: C, 68.08; H, 3.93. Found: C, 67.83; H, 3.69.

Cells and plasmids. Lung cancer cells H1299, HOP92, A549, 1-1460, prostate cancer cells PC-3, and primary lung fibroblast cells HEL and epithelial cells HBEC and BEAS-2B were from ATCC, MD; normal human prostate epithelial cells PrEC were from Lonza Inc., Allendale, N.J. PC-3 derivatives PC-3 MM2 were from Sue-Hwa Lin, M.D. Anderson Cancer Center, Houston, Tex. Par-4^{+/+} and Par-4^{-/-} MEFs were derived from wild type and Par-4-null C57/B6 mice generated by Taconic. Vimentin-null and wild type MEFs, as well as vimentin-expressing and vimentin-deficient SW13cells were from Anthony Brown (The Ohio State University, Columbus, Ohio).

Antibodies and siRNA duplexes. Par-4 (R334), CollA1 (H-197), vimentin (H-84) for Western blot; vimentin (RV202) for ICC; GRP78, CollA1 (H-197), and pan-cytokeratin (C11) antibodies were from Santa Cruz Biotechnology (Santa Cruz, Calif.). The β-actin antibody was from Sigma Chemical Corp. (St Louis, Mo.).

Co-immunoprecipitation and Western blot analysis. Protein extracted from cell lysates was filtered, pre-cleared with 25 μL (bed volume) of protein G-Sepharose beads and immunoprecipitated with 1 μg of respective antibodies. The eluted proteins were resolved by SDS-PAGE, and subjected to Western blot analysis as described previously. Bensch, et al. Nature 1968, 218(5147):1176-1177.

Apoptosis assays and detection of Cell Surface GRP78 Apoptotic nuclei were identified by ICC analysis for active caspase-3 or by DAPI staining. A total of three independent experiments were performed; and approximately 500 cells were scored in each experiment for apoptosis under a fluorescent microscope. Cell surface GRP78 expression on the cancer cell surface was quantified by FACS analysis in the Flow Cytometry Shared Resource Facility, Markey Cancer Center as previously described. Burikhanov, et al. Cell 2009, 138(2): 377-388; Burikhanov, et al. Cancer Res 2013, 73:1011-1019.

Animal experiments. Whole-blood samples and various tissues were collected from mice, 24 h after injection via the intra-peritoneal (i.p.) route with arylquins (10 mg/kg) or corn oil (100 μL) as vehicle control. Serum was separated from the blood samples and used for testing by Western blot analysis and ex vivo apoptosis as described previously. Burikhanov, et al. Cell 2009, 138(2): 377-388; Burikhanov, et al. Cancer Res 2013, 73:1011-1019.All animal procedures were performed with the Institutional Animal Care and Use Committee approval.

Computational Modeling. Molecular modeling of vimentin binding with arylquinoline, arylquinolones and arylthioquinolones was performed by using the previously reported computational protocol. Briefly, a ligand was docked into the binding cavity and the resulting poses were refined by molecular dynamics (MD)-simulations. The most favorable binding mode (with the lowest binding free energy), which was identified in the docking procedure, was subjected to an MD simulation for 1 as at 298 K and used in binding free energy calculations.

Statistical analyses. All experiments were performed in triplicate to verify the reproducibility of the findings. The results show a mean of at least 3 experiments±standard deviation. Statistical analyses were carried out with Statistical Analysis System software (SAS Institute, Cary, N.C.) and P values were calculated using the Student t test.

hERG Binding studies, An HEK-293 cell line stably expressing the hERG potassium channel (accession number U04270) referred to as hERG-HEK cells were received at passage 11 (P11) from Millipore (CYL3006, lot 2, Billerica, Mass.). [³H]-Dofetilide (specific activity of 80 Ci/mmol; labeled on the N-methyl group) was obtained from American Radiolabeled Chemicals (St. Louis, Mo.). Other chemicals and solvents were obtained from Sigma-Aldrich (Milwaukee, Wis.). Polyethylenimine (PEI) was obtained from Fluka/Sigma-Aldrich (St. Louis, Mo.), and Minimium Essential Medium (MEM) with GlutaMAX™ and phenol red, MEM non-essential amino acids solution (NEAA, 100×), G418 disulfate salt solution, fetal bovine serum (FBS), 0.05% Trypsin-EDTA 1× with phenol red, and Hank's balanced salt solution (HBSS) were obtained from Life Technologies (Carlsbad, Calif.).

hERG-HEK Cell culture. hERG-FIEK Cells were cultured according to the protocol provided by Millipore. Cells were maintained in MEM (with glutamax and phenol red) supplemented with 10% PBS, 1% NEAA and 400 geneticin, and incubated at 37 ° C. in a humidified atmosphere with 5% CO₂. Frozen aliquots of cells were transferred into T-75 cm² flasks and allowed to adhere for 4-8 h. The medium was replaced every 2 days. Passages were carried out at least 3 times after thawing at 6 day intervals. Cells were dissociated with trypsin/EDTA and seeded into new 150×25 mm dishes at 2-3×10⁶ cells per dish and placed at 30° C., 5% CO₂, for 40-48 h prior to membrane preparation. Membrane preparation occurred 6 days after the last passage (passage 20).

Membrane preparation. Cell membrane preparation was based on previous methods. See Herrmann, et al. J Mol Biol 1996, 264:933-953; Lavie, et al. J Org Chem 1965, 30(6):1774-1778; Chen, et al. A Practical Guide to Assay Development and High-throughput Screening in Drug Discovery, CRC Press Taylor and Francis Group, Boca Raton, Fla., 2010; Redfern, et al. Cardiovasc Res 2003, 58, 32-45. Cells were rinsed twice with HBSS at 37° C. and collected by scraping the dishes in 20 mL of ice-cold 0.32 M sucrose and homogenized on ice with a Teflon pestle using a Maximal Digital homogenizer (Fisher Scientific, Pittsburgh, Pa.) at 280 rpm for 30 sec. Homogenates were centrifuged at 300 g and 800 g for 4 min each at 4° C. Pellets were resuspended in 9 mL of ice-cold Milli-Q water and osmolarity restored by addition of 1 mL of 500 mM Tris buffer (pH 7.4) followed by suspension and centrifugation at 20,000 g for 30 min at 4° C. Pellets were homogenized in 2 mL assay buffer (50 mM Tris, 10 mM KCl, and 1 mM MgCl₂, 4° C.) and aliquots of cell membrane suspensions were stored at −80° C. and thawed the day of the [³H]-dofetilide binding assay. Protein content was determined prior to the assay using a Bradford protein assay with bovine albumin as the standard.

[³H]-Dofetilide binding assay. [³H]-Dofetilide binding assays using hERG-HEK293 cell membranes were based on previous methods. Redfern, et al. Cardiovasc Res 2003, 58, 32-45. Assays determining concentration-response were conducted in duplicate, and three independent assays were performed for each analog evaluated. Cell membrane suspension (5 pg) was added to duplicate tubes containing assay buffer, 25 μL, of a single concentration of arylquinoline (concentration range of 10 nM-100 μM for each experiment), and 25 μL of [³H]-dofetilide (5 nM, final concentration) for an assay volume of 250 μL. Binding occurred for 60 min at 25° C. and was terminated by rapid filtration through Whatman GF/B filters, which were pre-soaked in 0.25% PEI overnight, using a Brandel cell/membrane harvester (M-48; Brandel Inc,, Gaithersburg, Md.). Filters were washed three times with 1 mL of ice-cold assay buffer. Radioactivity was determined by liquid scintillation spectrometry using the Tri-Carb 2100-TR Liquid Scintillation Analyzer (Perkin-Elmer Life and Analytical Sciences)

[³H]-Dofetilide binding to plasma membranes overexpressing the hERG channel. [³H]-Dofetilide competition binding assays using HEK-293 cell membranes stably expressing the hERG channel (hERG-HEK) correlate well with results from voltage-clamp assays and provide useful predictive screening assays for QT prolongation.²⁸ We utilized a [³H]-dofetilide binding assay to evaluate arylquinoline interaction with hERG Amitriptyline (final concentration, 1 mM) was used as the positive control and exhibited an IC₅₀ value (10.7±2.25 μM) that was in agreement with published values. Jo, et al. Br J Pharmacol 2000; 129, 1474-1480, We selected a subset of the arylquins that possessed potent Par-4 secretory activity Concentrations of arylquins agents ranging from 10⁻⁹ to 10⁻⁴ M were assayed in duplicate for these experiments (n=3 experiments/analog). IC₅₀ values for hERG inhibition of [³H]-dofetilide binding ranged from 20-60 μM, Ratios of the IC₅₀ values for the hERG inhibition and IC₅₀ values for the inhibition of LS174T cell proliferation (Table 1) ranged from 2 to 4 orders of magnitude for the subset of arylquins that were studied.

Data analysis of [³H]-dofetilide binding. Arylquinoline, arylquinolone, and arylthioquinolone concentrations that produced 50% inhibition (IC₅₀) in the biological studies were determined from the concentration-response curves via the non-linear regression one-site competition-fitting program (Prism 5.04; GraphPad Software Inc. San Diego, Calif.)

As shown by the examples, targeting filament forming proteins by compounds that inhibit filament growth or hyperstabilize filaments is a successful strategy for the development of chemical probes and therapeutics. Among the most prominent examples of such compounds are swinholide, which inhibits actin filament growth inhibitor; taxol, which is a tubulin filament stabilizer; and vinblastine, which is a tubulin filament growth inhibitor. Both actin and tubulin are globular proteins as monomers, composed of both β sheets and α helices, and both contain cavities that are suitable for binding small molecules. A vimentin monomer is structurally distinct from actin and tubulin, because it possesses a linear rather than a globular structure, The N- and C-terminal regions of vimentin lack secondary structure, bit its core region, called the rod domain, consists of helices connected by flexible linker regions. In contrast to the relatively rigid actin and tubulin monomers, a vimentin monomer is predicted to have a fairly dynamic structure, which is not a classical druggable target. Two monomers of vimentin associate, however, into a more rigid, coiled-coil dimeric structure than the flexible monomer, the dimers form tetramers and, eventually, higher-order thick, elongated filament assemblies form, which are sixteen dimers thick. The exact structure of a fully-assembled vimentin filament remains unknown. This assembly, due to its well-featured surface topography, provides a suitable target for small molecule ligands. Although vimentin was touted as a potential target of anti-cancer agents due to its overexpression in various cancers,⁷ to our knowledge, only two families of compounds directly target vimentin: steroids such as withaferin A and 3-arylquinolines such as arylquinoline 1a. Withaferin A, a natural product isolated from Winter cherry, was demonstrated to modify covalently Cys328 in the 2B domain of vimentin. Withaferin A was proposed to bind in a shallow cleft on a surface of a tetramer formed by this domain in the vicinity of Cys328 and to trigger the observed aggregation of vimentin filaments. Computational modeling suggested that arylquinoline 1a bound this same site and triggered release of sequestered Par-4.

Initial SAR development focused on halogenation patterns within the C-3 aryl ring in arylquinoline 1a (Table 1) and followed the Par-4 secretory activity of these agents relative to vehicle when administered at 500 nM concentrations. Potency alone, however, is no longer sufficient to guide translational drug development. In developing these arylquinolines as potential drug candidates, we also sought arylquins that minimized activation of the hERG potassium channel associated with drug-induced, adverse cardiac events. We utilized a [³H]-dofetilide binding assay to evaluate the interaction of these arylquins with hERG and excluded for further development any arylquins with hERG IC₅₀ values less than 1 μM.

Arylquinoline 1a with an ortho-fluorophenyl group at C-3 was superior in potency to analogs lacking any substituents or to those with meta- and para-fluorophenyl groups 1d and 1f, respectively. In this study, we explored the introduction of other ortho-oriented halogens as well as multiple halogens. The ortho-chlorophenyl groups in 1b was comparable in activity to the ortho-fluorophenyl group in 1a, but the ortho-bromophenyl groups in 1c had diminished activity, Similarly, the meta- or para-chlorophenyl group in 1e and 1g, respectively, showed no improvement over the weakly active meta-or para-fluorophenyl groups in 1d and 1f, respectively. These SAR results clearly favored the presence of an ortho-fluorophenyl group in support of the computational model that suggested a crucial hydrogen bond between the ortho-fluoro group and Cys328. Presumably, halogens larger than a fluoro substituent encounter steric repulsions, and halogens at positions other than the ortho-position are unable to form the most stable, linear hydrogen bond between the fluorine and the sulfur of the Cys328 residue.

We explored the introduction of multiple halogens in the C-3 phenyl ring with a particular interest in determining if a second halogen would augment the Par-4 secretory activity seen with either an ortho-fluoro or chloro group. Synthesis and testing of the isomeric 2,3-, 2,4-, 2,5-, 2,6-, 3,4- and 3,5-difluorophenyl analogs 1h, 1j, 1k, 1m, 1n, and to revealed that one ortho-fluorine substituent in 1h, 1j and 1k was sufficient to produce a Par-4 secretagogue equipotent to the original arylquinoline 1a (Table 1). Two ortho-fluorine substituents as in the 2,6-difluorophenyl analog lm (Table 1) or the 2-chloro-6-fluorophenyl analog (Supplementary Material) did not lead to improved activity, and the lack of any ortho-fluorine substituent as in the 3,4- or 3,5-difluorinated analogs 1n and 1o had diminished activity relative to 1a.

The dichlorophenyl analogs were also evaluated and generally found to be less active than 1a. Only the 2,3-dichlorphenyl group in 1i displayed Par-4 secretory activity comparable to 1a, 1b and 1h. Finally, removal of all halogens or the introduction of electron-donating groups in the ortho-position led to arylquins with only minimal secretory activity(Supplementary Material). Other electron-withdrawing substitutents, such as the ortho-cyano or ortho-carboxy groups, led to the biologically inactive tetracyclic arylquins, dibenzo[b,f][1,8]naphthyridine 6 or dibenzo[b,f][1,8]naphthyridin-5(6H)-one (7), respectively (FIG. 3). Another tetracyclic family of arylquins, the imidazo[1,2-a]quinolines 8a and 8b, also had little Par-4 secretory activity (data not shown). In summary, a detailed exploration of substituent patterns in the C-3 aryl group in arylquins revealed the preference of a C-3 ortho-fluorophenyl or ortho-chlorophenyl substituent as in arylquins 1a, 1b, 1h and 1i. No particular advantage accrued to the introduction of additional halogens or other types of substituents, and we retained the C-3 ortho-fluorophenyl group in subsequent SAR studies involving variations in substituents on the quinoline ring.

We next explored modifications of the C-2, C-5, C-6 and C-7 positions on the quinoline ring (Table 2) within the arylquinoline platform with a particular interest in replacing the C-2 amino group in arylquinoline 1a. Computation modeling, which suggested a hydrogen bond between this amino group and Asp331 in vimentin, hinted that other heteroatoms than nitrogen might suffice at the C-2 position and that loss of a heteroatom substituent at C-2 would diminish activity. Consistent with these predictions, reduction at C-2 to the unsubstituted arylquinoline diminished Par-4 secretory activity, and arylquins with C-2 sulfur substituents proved to be biologically active. In fact, the installation of biotin group connected through a C-2 thioether linkage proved invaluable in providing a biologically active biotinylated arylquinoline that was used in pull-down studies to identify vimentin as the binding target.

Condensation of substituted 2-amino-4-(N,N-dimethylamino)benzaldehyde (5) with α-arylacetyl chlorides provided access to the 3-arylquinolones 9, and treatment of 6 with Lawesson's reagent provided the corresponding 3-arylquinothiones 10 (FIG. 4). Both 9 and 10 possessed diminished Par-4 secretory activity relative to arylquinoline 1a. Replacing the C-2 amino group in 1a with chloro group produced an arylquinolone 12 with activity comparable to but not exceeding that of arylquinoline 1a. On the other hand, replacing the C-2 amino group with a thiomethoxy, N-methylpiperazinyl, and morpholino group in arylquins 11, 13a and 13b, respectively, led to diminished activity to varying degrees. In summary, with respect to the C-2 position, only the introduction of chlorine provided an arylquinolone with potentially interesting Par-4 secretory activity, and the elevated hERG activation seen with arylquins 9-12 (Table 2) makes these analogs more attractive than the original C-2 amino group in 1a.

We also modified the C-5, C-6 and C-7 positions in arylquinoline 1a to determine the secretory activity for the C-7 N,N-dimethylarnino group and if other substituents in this quadrant supported enhanced secretory activity. Computational modeling revealed that the arylquinoline 1a as well as arylquins 15a-15h had similar binding modes with vimentin in terms of the orientation of the arylquins in the binding site. Computational modeling also suggested that the two methyl groups in the C-7 N,N-dimethylamino group participated in weak van der Waals interactions with the hydrophobic Leu326 and Va1330 residues in vimentin. Friedlander condensations of 2-aminobenzaldehydes 14 readily furnished arylquins modified at C-5, C-6 and C-7 (FIG. 2) and devoid of the C-7 N,N-dimethylamino group. Removing the C-7 AN-dimethylamino group without introducing any other substituents, as in arylquinoline 15a, decreased Par-4 secretory activity only slightly relative to the parent arylquinoline 1a, consistent with the weak van der Waals interactions seen in computational modeling. Removing the C-7 N,N-dimethylarnino group and introducing other substituents at C-5, 6, 7 and 8 of the arylquins was also of relatively little consequence in terms of potency. Introduction of either a 5-fluoro group in 15b or 6-chloro group in 15e group produced analogs equipotent to 15a. Introduction of either a 5-chloro group in 15c or a 5-bromo group in 15d eliminated the hydrogen bond with Thr327 side-chain and accounted for the lower Par-4 secretory activity seen in 15e and 15d than that in arylquinoline 1a. Introduction of a 7-chloro group in 15f, a 7-carboxymethyl group in 15g, or a 7-carboxylate group in 15h eliminated the hydrogen bond with Asp331 side-chain and explained the reduced activity of these arylquins. These minimal increases in Par-4 secretory activity coupled with hERG values (Table 2) that were greater than arylquinoline 1a by a factor of two to three makes arylquins attractive.

In summary, experimental evaluation of the Par-4 secretory activity of arylquins supported computational findings, Three factors dominated the relative secretory activities of these arylquins: [1] the relative strengths of hydrogen bonds between C-2 amino groups and the side-chain of Asp331, [2] the relative strengths of hydrogen bonds between the quinoline nitrogen and the side-chain of Thr327, and [3] the relative strengths of hydrogen bonds between the ortho-fluorine groups in the C-3 aryl groups and the side-chain of Cys328. It remains unclear whether arylquinoline, arylquinolone or arylthioquinolone binding causes filament aggregation, disruption or steric blockade of the Par-4 binding site, and future structural work will address these vimentin interactions and guide rational SAR optimization,

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A compound according to formula (I): 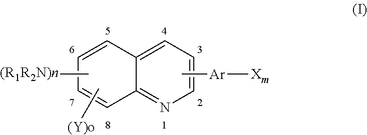
or a pharmaceutically acceptable salt thereof;
wherein n is 1, 2, or 3, for each NR₁R₂, R₁ and R₂ are independently H, alkyl, alkoxy, aryl, heteroaryl; Ar is aryl or heteroaryl, which can be further substituted with halogen, amino, alkylamino, dialkylamino, arylalkylamino, N-oxides of dialkylamino, trialkylammoniurn, mercapto, alkylthio, alkanoyl, nitro, nitrosyl, cyano, alkoxy, alkenyloxy, aryl, heteroaryl, sulfonyl, sulfonamide, CONR₃R₄, NR₃CO(R₄), NR₃COO(R₄), NR₃CONR₄R₅ where R₃, R₄, R₅, are independently, H, alkyl, aryl, heteroaryl or a fluorine; X represents halogen; m is 1, 2, 3, 4, or 5; o is 1, 2, 3 or 4; and each Y is independently a halogen, alkoxy, alkylthio, a substituted or unsubstituted heterocycle.

2. The compound of claim 1, wherein Ar is a heteroaryl.

3. The compound of claim 1, wherein Ar is pyridinyl, diazinyl, pyrimidinyl, oxazolyl or imidazolyl.

4. The compound of claim 1, wherein Ar is phenyl.

5. The compound of claim 1, wherein n is 1 or 2; for each NR₁R₂, R₁ and R₂ are independently H or a lower alkyl; m is 1 to 3 and X is selected from fluorine or chlorine, o is 1; and Y is chloro, fluoro, a C₁-C₆ alkoxy, a C₁-C₆ alkythio or a substituted or unsubstituted nitrogen-containing heterocycle.

6. The compound of claim 1, wherein the substituent at the C-2 position of the quinoline ring is either NR₁R₂, or Y, the substituent at C-3 is phenyl or pyridinyl, diazinyl, pyrimidinyl, oxazolyl or imidazolyl; the substituent at C-4 is H; the substituent at C-5 is H or Y; the substituent at C-6 is H or Y; the substituent at C-7 is H, NR₁R₂, or Y; the substituent at C-8 is H; provided that there is at east one NR₁R₂ at C-2 or C-7 and at least one Y at C-2, C-5, C-6 or C-7.

7. A pharmaceutically acceptable composition comprising a compound of claims 1 or a pharmaceutically acceptable salt thereof and a pharmaceutically acceptable additive.

8. A method for screening for compounds that inhibit vimentin binding to PAR-4 or that dissociate vimentin-PAR-4 complexes, comprising exposing a sample comprising cells that express vimentin to various concentrations of PAR-4 in the presence of a compound of claim 1 and detecting the level of vimentin-PAR-4 complex formation.

9. A compound for use in the treatment of cancer in a subject in need thereof comprising administering to the subject an effective amount of a compound of claim 1 or pharmaceutically acceptable salt thereof or a pharmaceutical composition thereof.

10. The compound of claim 9, wherein the cancer treated is selected from the group consisting of colorectal cancer, prostate cancer, brain cancer, liver cancer, breast cancer and lung cancer.
